(19) United States
(12) Patent Application Publication
(10) Pub. No.: US 2003/0152927 A1
(43) Pub. Date: Aug. 14, 2003

(54) Closed substrate platforms suitable for analysis of biomolecules

(76) Inventors: Mogens Havsteen Jakobsen, Vanlose (DK); Lars Kongsbak, Holte (DK)

Correspondence Address: Peter F. Corless, Edwards & Angell, LLP, P.O. Box 9169, Boston, MA, 02209, US

(21) Appl. No.: 10/032,381

(22) Filed: Oct. 25, 2001

Related U.S. Application Data
(60) Provisional application No. 60/243,349, filed on Oct. 25, 2000.
(60) Provisional application No. 60/305,726, filed on Jul. 16, 2001.

(51) Int. Cl. C12Q 1/68

(52) U.S. Cl. 435/6

(57) ABSTRACT

The invention relates to novel analysis slides or substrates with a closed configuration. The slides may be used for any application which normally utilizes a conventional microscope slide and can be used in conjunction with any type of equipment typically used to manipulate or evaluate a standard microscope slide. In particular, the invention provides closed slides for covalent immobilization of polypeptides and nucleic acids. Further provided are methods for carrying out biological assays using arrays of biomolecules immobilized on the slides of the invention. The arrays of the invention are comprised of a unique meandering or straight channel design as compared to the rectangular design of conventional arrays.

The present application claims the benefit of U.S. Provisional Application Serial No. 60/243,349 filed Oct. 25, 2000, and U.S. Provisional Application Serial No. 60/305,726 filed Jul. 16, 2001, both of which applications are incorporated herein by referenced in their entirety.

### BACKGROUND OF THE INVENTION

1. Field of the Invention

The invention relates to novel platforms, particularly slides and compartments such as microscopic slides, of closed configurations. The slides may be used for any application which normally utilizes a conventional microscope slide and can be used in conjunction with any type of equipment typically used to manipulate or evaluate a standard microscope slide. In particular, the invention provides for closed slides for covalent immobilization of biomolecules, e.g. peptides, polypeptides, nucleic acids, nucleic acid binding partners, proteins, receptors, antibodies, enzymes, oligo saccharides, polysaccharides, cells, arrays of ligands (e.g. non-protein ligands), and the like. Further provided are methods for carrying out biological assays using arrays of biomolecules immobilized on the slides of the invention.

2. Background

The development of bio-array technologies promises to revolutionize the way biological research is carried out. Bio-arrays, wherein a library of biomolecules is immobilized on a small slide or chip, allow hundreds or thousands of assays to be carried out simultaneously on a miniaturized scale. This permits researchers to quickly gain large amounts of information from a single sample. In many cases, bio-array type analysis would be impossible using traditional biological techniques due to the rarity of the sample being tested and the time and expense necessary to carry out such a large scale analysis.

Although bio-arrays are powerful research tools, they suffer from a number of shortcomings. For example, bio-arrays tend to be expensive to produce due to difficulties involved in reproducibly manufacturing high quality arrays. Also, bio-array techniques can not always provide the sensitivity necessary to perform a desired experiment. Therefore, it would be desirable to provide an improved platform for the production of arrays which results in a less expensive, more reproducible and more sensitive bio-array.

There are two fundamentally different approaches to the manufacturing of bio-arrays: 1) “in situ synthesis” and 2) “micro spotting”. The in situ synthesis approach involves monomer-by-monomer synthesis directly on the substrate carrier. This approach has some inherent drawbacks as the synthesis of oligomers includes many chemical steps which never provide 100% yield. Thus, bio-arrays produced via the in situ synthesis strategy generally contain truncated sequences leading to differences in the composition from array to array. The micro spotting approach involves dispensing of biomolecules onto the substrate carrier followed by immobilization of the molecules onto the surface. This approach offers the advantage that materials can be obtained from natural sources, or synthesized on standard synthesizers, purified and characterized prior to construction of the array. Thus, bio-arrays produced by the micro spotting approach generally are more reproducible and of higher quality than bio-arrays produced by the in situ synthesis approach.

### SUMMARY OF THE INVENTION

The present invention provides novel substrate analysis platforms that can be employed in a variety of scanning or analysis apparatus, including applications or instruments which normally employs a standard microscope slide. A preferred use of the platforms is the immobilization of biomolecules for investigation of biomolecule interactions. The unique meandering design of the array allows for use of reduced volumes of sample and buffers as compared to conventional arrays.

In a first embodiment, a slide article, preferably rectangular and plastic, provides for a closed substrate platform with a “meandering design”. The design of the analysis platform allows for liquid sample, injected through a narrow inlet port connected to a narrow tube, to enter the wider meandering channel analysis chamber in the absence of air bubbles and simplifying the transition of the liquid sample from a narrow channel to a wider channel. The meandering design of the analysis channel chamber allows for the liquid sample to travel with an even liquid front velocity. The pressure applied by a user or automated system allows for the liquid sample to be distributed evenly through the analysis area.

In a second preferred embodiment, the meandering design of the analysis chamber provides for a reduced volume of liquid sample used for analysis as the meandering design provides for a large surface area requiring a low volume of sample for hybridization, as compared to conventional open microarrays. Having a low volume chamber offers the possibility of having high concentrations of target DNA.

In a third preferred embodiment, a slide article, preferably rectangular and plastic, provides for a closed substrate platform with a “straight canal”. The design of the analysis platform allows for liquid sample, injected through a narrow inlet port connected to a narrow tube. The inlet port is comprised of an adapter which fits into the inlet port and is conical in shape. The top part of the adapter is constructed to receive a pipette or a syringe needle. The adapter is comprised of rubber or a silicon-based material so that there is a tight contact between the pipette tip or syringe needle when fluid is delivered. The fluid travels from the adapter to the analysis area through a canal which is interrupted by a flow restrictor. The flow restrictor serves the purpose of allowing for the liquid sample to be distributed evenly through the analysis area when pressure is applied by a user or automated system. The analysis area is preferably a straight and narrow canal with no turns. At a defined area, probes (discussed infra) are attached to the bottom of the canal. The fluid travels from the analysis area through a canal and arrives at a buffer chamber. Connected to the buffer chamber is a short capillary canal that opens into a meandering design waste area. The waste area ends in a vent, shaped as a capillary chamber.

In another embodiment, a slide article or substrate analysis platform comprising shallow depressions on the top and bottom surfaces is provided. The depression on the top surface provides a well capable of containing a specific volume of liquid. The depression on the bottom surface prevents the slide from becoming scratched during handling. The slide preferably contains paired finger indentations to aid in removal of the slide from a flat surface. The slide is preferably used in conjunction with a coverslip which is capable of sealing the opening of the well on the top surface of the slide due to hydrophilic interactions. The area outside the depression on the top surface is preferably frosted, so as to provide a clear identification of the hybridization area and the remaining “handling area”.

In another preferred embodiment, the slide is comprised of a bottom surface plastic structure as described above. The top surface of the slide is comprised of a thin plastic film or laminate which is placed over the bottom part of the slide and sealed using heat or adhesive followed by physical pressure to ensure airtight sealing and prevent any liquid or gas from escaping through the seal. This has the advantage of providing for a thin slide that can be used in many applications or instruments which normally employ a standard microscope slide.

In another preferred embodiment, a closed slide is provided wherein the substrate is enclosed within a container that is preferably sealed. The container provides ports for introduction of fluid into the container and venting of air out of the container. The ports connect to an integrated microfluidics system that permits sample loading and buffer washing without opening the sealed container. An outlet port and waste area within the container are also provided for expelling and containing waste materials.

The slides are preferably constructed of a polymer with low intrinsic fluorescence emission. Preferably the polymer is resistant to extremes of temperature (high and low), sonication and a wide variety of solvent conditions, such as extremes of pH, high ionic strength or organic solvents. Preferred polymers include polycarbonate, Topas (trade name; available from Hoechst). Other suitable materials for construction of the analysis platforms of the invention include e.g. polyethylene, polypropylene, polystyrene, polymethylacrylate, and the like.

Slides of the invention may be used for any type of application which may be carried out using a standard microscope slide. For example, the slides may be used for microscopic analysis of samples, smears, sections, etc. Other types of applications include e.g. diagnostics; SNP analysis; gene expression including e.g. detection of intron/exon splicing, and to evaluate if expression of certain genes is modulated by drug candidates); toxicology studies including toxicology on cells; protein-to-protein interactions; plant and animal breeding studies; environmental studies; and the like.

Slides or analysis platforms of the invention may be suitably used in conjunction with any type of a wide variety of analysis equipment, materials or reagents, including equipment, materials and reagents used with standard microscope slides, such as e.g. coverslips, slide washers, pipettors, inkjet printers or spotters, or robotics systems. Additionally, the slides or analysis platforms of the invention may be analyzed using any type of instrument or device capable of analyzing or reading a standard microscope slide including, for example, microscopes, scanners, readers, imagers, or the like.

The invention also provides immobilized biomolecules on the surface of the substrate. Preferably, nucleic acid, nucleic acid binding partners, proteins, antibodies, polysaccharides or polypeptides are immobilized in an array wherein each unique sequence is located at a defined position on the substrate. The arrays preferably contain at least about 10 to about 100 unique sequences per cm². Immobilized nucleic acids preferably contain from about 2 to about 5000 nucleotides, more typically 2 to about 1000 nucleotides, and polypeptides preferably contain from about 2 to about 5000 amino acids.

Immobilized nucleic acid chains of the invention preferably contain at least one LNA nucleoside analogue. LNA nucleoside analogues are disclosed in WO 99/14226. Also provided are oligomers composed entirely of LNA nucleosides. Immobilized nucleic acids may be either single stranded or double stranded.

Biomolecules are preferably immobilized onto the substrate using a photochemical linker, preferably a photoreactive linker, such as a photoreactive ketone, or particularly a photoreactive quinone such as disclosed in WO 96/31557. Also provided are flexible linkers which can serve as a spacer between the substrate surface and the biomolecule. Nucleic acid, polysaccharide and polypeptide chains are preferably immobilized via one end of the chain.

The invention also provides methods for carrying out biological assays using the substrate platforms and fluidic devices of the invention. A wide variety of assays may be carried on the analysis platforms and fluidic devices of the invention, including any type of assay which may be carried out using a standard microscope slide.

Specific examples include assays wherein one component is immobilized on the surface of the slide. Preferred assays involve immobilized arrays of polypeptide or nucleic acid sequences which may be exposed to a biomolecule (i.e. a nucleic acid, polypeptide, hormone, small molecule drug or drug candidate, etc.) under conditions which favor interaction between the biomolecule and the immobilized molecules. Preferably, interactions between the molecules are detected by virtue of a detectable feature on the biomolecule, e.g. a chemoluminescent tag such as a radiolabel (e.g. ¹²⁵I, tritium ³²P, ⁹⁹Tc, and the like); fluorescent tag; or an inducible tag e.g. a functional group that is activated by energy input such as electric impulse, radiation (e.g. UV radiation); and the like. The methods of the invention may be used e.g. to investigate interactions between nucleic acid-nucleic acid, nucleic acid-polypeptide, polypeptide-polypeptide, etc. Particularly preferred assays which may be performed using the methods of the invention include gene expression profiling; immunoassays; diagnostics; SNP analysis; gene expression including e.g. detection of intron/exon splicing, and the like.

Slides or analysis platforms of the invention may also be used for applications or assays not involving immobilized biomolecules.

Other aspects of the invention are disclosed infra.

### BRIEF DESCRIPTION OF THE DRAWINGS

FIG. 1 shows a plan view of the general form of a preferred embodiment of a meandering design closed substrate platform for immobilization of biomolecules.

FIG. 2 shows section views and top and bottom views of different parts of the closed slide as shown in FIG. 1.

FIG. 3 shows a plan view of the meandering design of the analysis chamber for immobilization of biomolecules.

FIG. 4 shows a plan view of the hydrophobic stop located at the end of the analysis chamber and a plan view of the structure interfacing from the microstructures to the waste chamber located downstream from the hydrophobic stop.

FIG. 5 shows a plan view of the general rectangular form of the closed substrate platform for immobilization of biomolecules.

FIG. 6 shows a longitudinal cross-section of the closed slide as shown in FIG. 5.

FIG. 7 shows a plan view of an embodiment of the rectangular closed substrate platform for immobilization of biomolecules.

FIG. 8 shows a longitudinal cross-section view of the closed slide substrate platform as shown in FIG. 7.

FIG. 9 shows a plan view of another embodiment of the closed slide substrate platform for immobilization of biomolecules.

FIG. 10 shows a longitudinal cross-section view through the center of the closed slide substrate platform as shown in FIG. 9.

FIG. 11 shows a longitudinal cross-section view through the buffer vent of the closed slide substrate platform as shown in FIG. 9.

FIG. 12 shows a longitudinal cross-section view through the sample vent and waste outlet of the closed slide substrate platform as shown in FIG. 9.

FIG. 13 shows a top view of the outside of the closed slide substrate platform as shown in FIG. 9 wherein a cover clip has been placed over the buffer inlet.

FIG. 14 shown a cross sectional view of closed slide and cover clip as shown in FIG. 13.

FIG. 15 shows a top view of the outside of the closed slide substrate platform as shown in FIG. 9 wherein a cover clip has been placed over the sample inlet.

FIG. 16 shows a cross sectional view of closed slide and cover clip as shown in FIG. 15.

FIG. 17 shows a further view of the cover clip of FIGS. 15 and 16.

FIG. 18 shows a top view of the inlet port and adapter and a side view of the inlet port.

FIG. 19 shows a top view of a preferred embodiment of a straight channel substrate analysis platform. The lower part of the diagram shows a top view of the capillary tubes and chambers preceding the meandering design waste area.

FIG. 20 shows the top view of the portions of the substrate analysis platform.

### DETAILED DESCRIPTION OF THE INVENTION

The present invention provides for closed substrate platforms which are a significant improvement over standard microscope slides. The substrate platforms are preferably used for the immobilization of biomolecules, but may be used for any application normally utilizing a microscope slide.

As used herein the term “substrate platform”, “analysis platform”, “hybridization chamber” or “slide element” or similar term refers to the foundation upon which biomolecules may be immobilized, samples may be applied for analysis or biological assays may be carried out. The terms “substrate platform”, “fluidic device”,“microfluidic structure”, “analysis platform”, “hybridization chamber”, “slide element” and ‘slide’ or “microscope slide” may be used interchangeably, however, where applicable, the term substrate platform refers to the part of the slide to which the sample is applied and the term slide refers to the entire structure including the substrate platform.

As used herein, “microfluidic” refers to the volumes of sample that can be used in the analysis platform, for example at least about 1:1 to at least about 30:1.

As used herein the term “microscope slide” or “standard microscope slide” refers to any type of slide which falls within the parameters recognized in the art. For example, in the United States, typical slide elements have dimensions of 1 inch×3 inches. In Europe, typical slide dimensions include 25 mm×75 mm, or 26 mm×76 mm. Typical slide thickness are from about 1 mm to about 1.3 mm.

As used herein the term “meandering design” refers to the shape of the substrate analysis chamber which is characterized by an “S-like” shape wherein a series of straight parallel tubes end in 180° semi-circular turn leading into another straight tube running parallel to the previous tube, and so forth, thereby forming the meandering design.

As used herein, the term “straight channel” refers to the shape of the substrate analysis platform which is characterized by a straight tube.

As used herein, the term “airtight” refers to the sealing of the top and bottom sections of the substrate platform such that air or fluids cannot leak through the seal.

The substrate platform may be constructed from a variety of materials such as plastics, quartz, silicon, polymers, gels, resins, carbon, metal, membranes, glass, etc. or from a combination of several types of materials such as a polymer blend, polymer coated glass, silicon oxide coated metal, etc. Particularly preferred substrate materials are polymers which contain a low intrinsic fluorescence emission, such as polycarbonate, Topas (trade name; available from Hoechst), polymethyl methacrylate (PMMA), and the like.

The term “plastics” as used herein refers to polymers, such as thermoplastic polymers. The plastic is used in the manufacture of microfluidic devices. Such devices include, but are not limited to:miniature diagnostic systems for biopharmaceutical applications, miniature devices for directing fluid flow, miniature sensor devices for pharmaceutical and biochemical applications, and three-dimensional microfluidic systems. When used in these applications, it is preferred that the plastic is selected from the group consisting of homopolymers and copolymers of polycarbonate, polystyrene, polyacrylic, polyester, polyolefin, polyacrylate, and mixtures thereof.

The term “clarity” as used herein, is the degree of absence of impurities which may impair the passage of light through the slide and is measured by the amount of light that can pass through the slide, measured at a wavelength of preferably 530 nm. The amount of light passing through the slide is preferably at least 75% of total light from the light source, more preferably 85%, most preferably 90%.

The term “low intrinsic fluorescence” as used herein refers to a material or substrate which emits less than about 50 percent of the detected signal of a test sample on the substrate, thereby providing a signal: noise ratio at detection levels of 2:1.

As used herein, the term “depression” refers to an indentation on the surface of the substrate analysis platform, wherein the indentation can be square or rectangular and the sides of the indented portion are either perpendicular to the indented surface or angled by at least 50° relative to the indented surface.

Particularly preferred are slides that have a flatness of less than or equal to about 20 μm, wherein the flatness does not deviate on a slide and between slides, more than 1 μm per millimeter. Preferably the slide has a roughness of about an RA of less than about 100 nm, preferably an RA of less than about 50 nm, more preferably an RA of less than about 20 nm.

Preferably, the substrate platform is constructed of a material that is capable of covalently binding to a biomolecule without activating the surface of the platform. For example, the substrate material may provide reactive groups at the surface such as carboxyl, amino, hydroxyl, sulfhydryl, etc. Alternatively, the surface of the substrate may be derivatized so as to provide functional groups which will allow covalent attachment of a biomolecule. For example, the substrate may be derivatized with silanes or other chemical groups; or the substrate may be surface modified such as by plasma treatment and the like; etc.

Preferably the surface of the substrate platform is substantially smooth so as to allow uniform binding of biomolecules and effective analysis of molecules bound to the substrate using a variety of scanners, readers, detectors, etc. Alternatively, the surface of the substrate may be treated or coated so as to increase the binding capacity of the substrate. For example, a greater surface area for biomolecule binding may be achieved by roughening the surface of the substrate or by coating it with gel, particles, beads, etc. Preferably the substrate platform is optimized so as to provide the greatest binding capacity while still allowing efficient manipulation and evaluation of biomolecules bound to the surface.

The substrate platform is preferably constructed of materials which are resistant to extremes of low and high temperatures, i.e. temperatures of −5° C. to +105° C.; resistant to extremes of low and high pH, i.e. pH over a range of 1 to 13; resistant to sonication; and resistant to a wide variety of solvent conditions, i.e. high ionic strength and organic solvents such as ethanol, methanol, formamide, DMSO, etc. Particularly preferred substrate platforms are resistant to thermocycling such as performed during PCR. The substrate platforms are preferably resistant to multiple, i.e. about 10 to about 50 rounds of heating and cooling, such as would be obtainable with an art recognized thermocycler.

By the term ‘resistant’ it is meant that the fundamental shape and properties of the substrate platform are not altered in a way which will affect the performance or functionality of the platform. For example, resistance is meant to indicate that exposure to an extreme temperature or pH will not cause the platform to melt, warp, etc. and that the platform will still be capable of covalently binding a biomolecule to the surface after such exposure.

The substrate platform may be constructed in a variety of shapes and sizes so as to allow easy manipulation of the substrate and compatibility with a variety of standard lab equipment such as microtiter plates, multichannel pipettors, microscopes, inkjet-type array spotters, photolithographic array synthesis equipment, array scanners or readers, fluorescence detectors, infra-red (IR) detectors, mass spectrometers, thermocyclers, high throughput machinery, robotics, etc. For example, the substrate platform may be constructed so as to have any convenient shape such as a meandering design, square, rectangle, circle, sphere, disc, slide, chip, film, plate, pad, tube or channel, strand, box, etc.

Preferably, the substrate platform is substantially flat with optional raised, depressed or indented regions to allow ease of manipulation. For example, the edges of the substrate platform may contain finger indents or ridges to facilitate handling and/or the surface may contain one or more wells which are capable of containing a specific volume of fluid. Particularly preferred substrate platforms are constructed in the general size and shape of a microscope slide and are compatible with any type of instrument that is capable of manipulating or evaluating a microscope slide.

Particularly preferred substrate platforms are comprised of a meandering design comprising at least about one U-shaped meandering substrate platform, specifically, two parallel tubes connected via a 180° semi-circular end, more preferably at least about three parallel tubes connected via a 180° semi-circular end, most preferably at least about ten parallel tubes connected via a 180° semi-circular end. In a preferred embodiment the substrate analysis platform is comprised of five parallel tubes connected via a 180° semi-circular end.

In a most preferred embodiment, the design of the hybridization area is comprised of a straight narrow channel from inlet to outlet port without any 180° semi-circular turns. The substrate platform is comprised of at least about one straight narrow channel from the inlet to the outlet port, more preferably the substrate platform is comprised of about, three straight narrow channels, each with their own inlet ports, most preferably more preferably the substrate platform is comprised of about five straight narrow channels, each with their own inlet ports. According to the present invention one straight narrow hybridization platform is sufficient, however more than one substrate platform may be used, each separate from the other and with each substrate platforms having individual inlet and outlet ports so that there is no cross-contamination between the samples.

The substrate platform may contain one or more typically a plurality of channels or tubular sections that provide for flow and residence of test samples. For instance, the closed configuration systems of the invention suitably may have flow channels for transport and analysis of a test sample. The substrate platform also typically has one, or a plurality of analytical areas. Such distinct analytical areas may reside e.g. in a test area of an open system of the invention, where each area is defined by a defined line, channel or the like in the substrate platform surface.

The substrate platform may be constructed in a variety of colors or with a variety of markings which perform both decorative and/or functional purposes. For example, the substrate platform may be constructed of materials containing dyes or pigments to provide a colored product. The color can serve as a means of identification or may serve to reduce the intrinsic fluorescence of the substrate material. Additionally, the substrate may be clear or opaque. Preferably, the substrate material is clear so as to allow light to pass through the substrate platform. In another aspect of the invention, the substrate platform may contain markings such as numbers, words, pictures, company logos, etc. In a particularly preferred embodiment, the substrate platform contains a bar code to allow unique identification of individual platforms.

Markings on the substrate platform may be made by any art recognized method including, for example, application of stickers or other adhesives; application of ink directly onto the substrate surface by a well-defined deposit e.g. an inkjet printer, a pin-spotter, etc.; raised or indented regions formed during the molding of the substrate platform; etched or frosted areas added after molding of the substrate platform; etc. Preferably, the markings are located outside the area to be used for sample analysis and may serve to demarcate the sample analysis area.

The substrate platforms of the invention may be constructed by any of a variety of methods, e.g. injection molding, hot embossing, mechanical machining, etching, with injection molding being generally preferred.

Substrate platforms of the invention may be constructed in closed configuration. By ‘closed configuration’ it is meant that the substrate is enclosed within a sealed container and has integrated microfluidic structures for sample loading and washing.

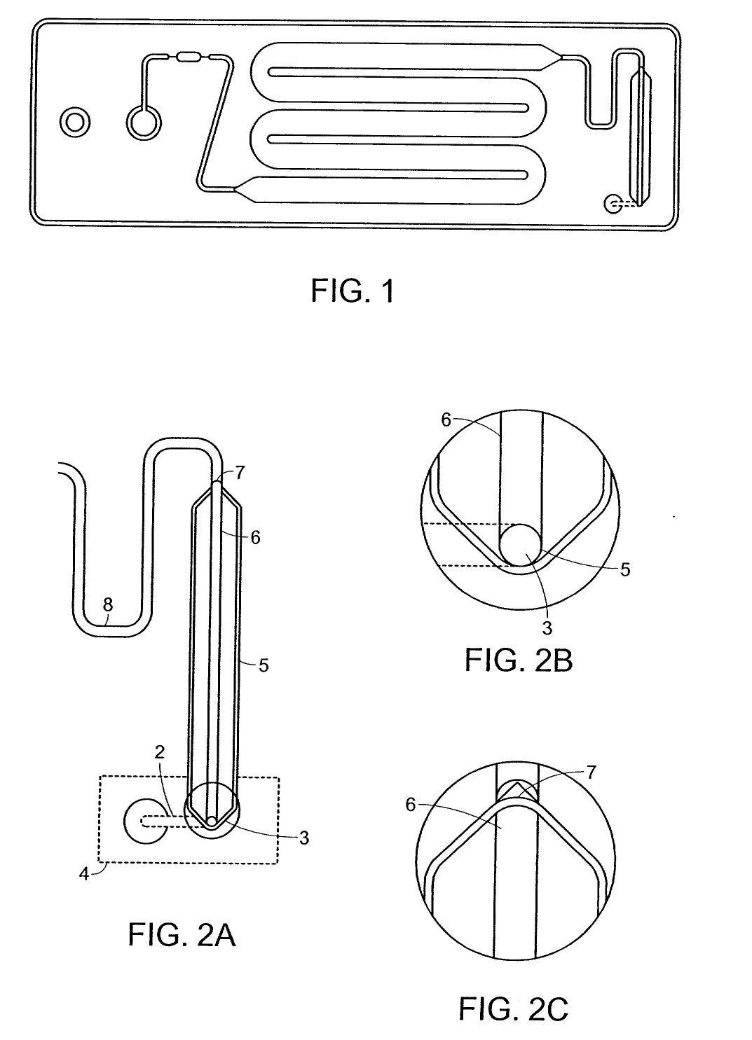

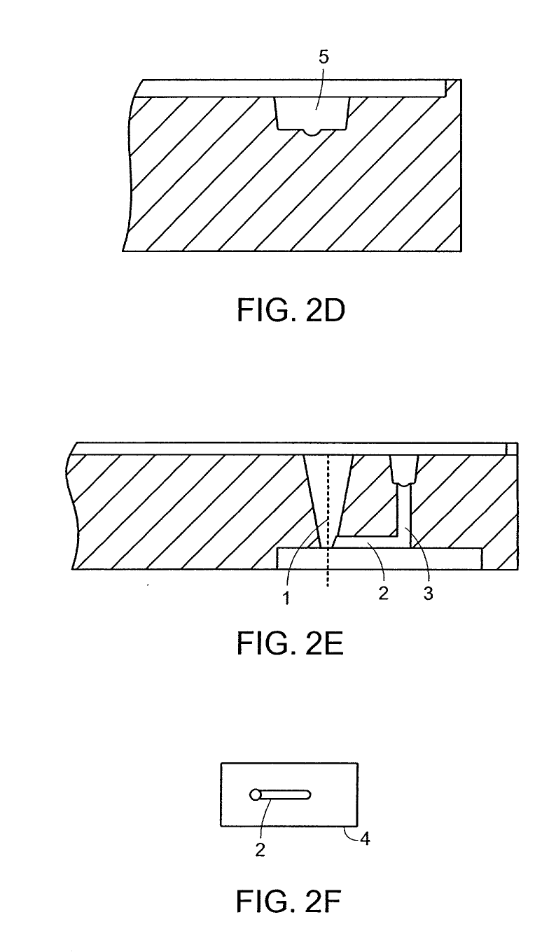

A preferred closed substrate platform is of a meander design as shown in FIG. 1. According to the invention, the sample or buffer is loaded by inserting a pipette into the sample port, which almost traverses the full thickness of the closed slide as illustrated in FIG. 2E. The sample port is, preferably conical to ensure a tight fit to the tip of the pipette 1. The sample or buffer runs via a small channel 2 to a position directly beneath the inlet chamber and further upwards via a small channel 3 to the bottom end of this chamber 5. The U-shaped inlet causes a pressure drop over the inlet structure from the pipette to the inlet chamber and this reduces the dependence of the user-applied pressure on the velocity of the liquid front in the analysis chamber.

The design as described above, is superior to conventional bioarrays as the meandering shape is conforms with properties of fluids, such as surface tension, front-velocity, etc and thus enhances the reliability and reproducibility of liquid addition, and decreases the risk of introducing air bubbles.

The inlet chamber 5, illustrated in FIG. 2, is long and narrow to ensure that liquid injected into the chamber is in contact with all walls of the chamber and thus pushing any existing bubbles forward to the end of the chamber and out through a small channel 8 connecting the inlet chamber 5 with the analysis chamber 9.

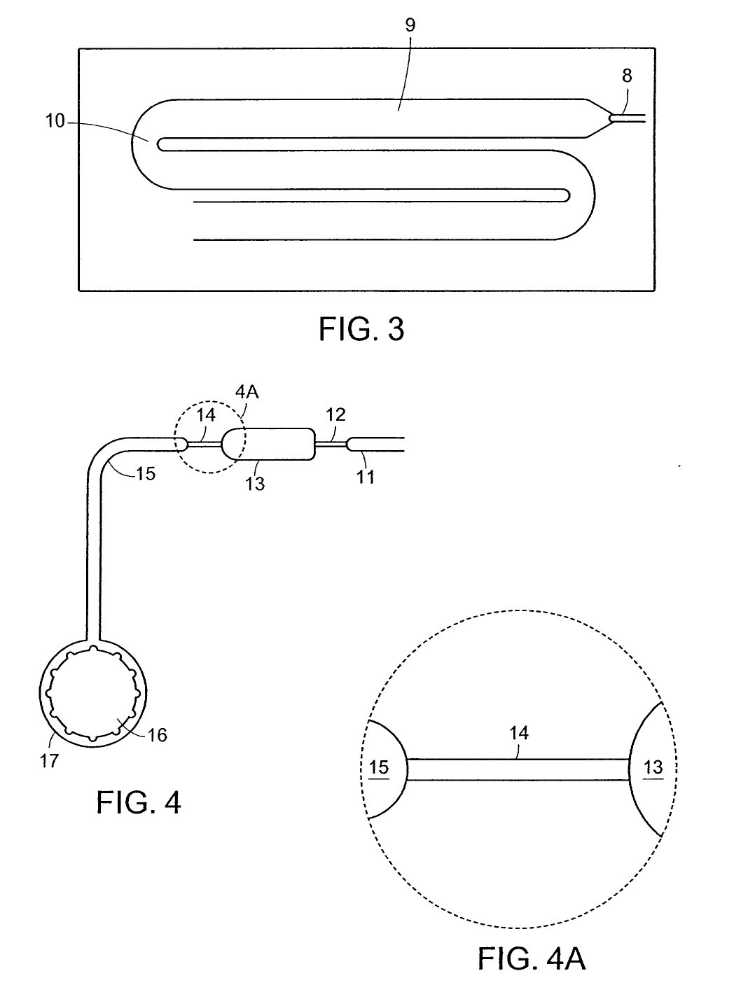

The analysis chamber is most preferably a channel meandering up and down the slide. The pressure applied by the user causes the sample or buffer to distribute evenly through the analysis area. In the 180° turns of the analysis area 10 (see FIG. 3) the path taken by the liquid at the outer part of the curve is longer than the path taken by the liquid at the inside of the turn. Therefore the outer liquid needs to travel faster than the inner liquid to obtain an even liquid front velocity across the width of the channel. This is implemented by reducing the width of the meandering channel from at least about 3 mm to at least about 2 mm in the turns, thus reducing the differences in travel distances and therefore the differences in flow velocities. The widths of the meandering channel compared to the widths of the turns can be varied as long as the liquid front-velocity remains even.

As used herein, “even liquid front velocity” refers to the even rate of movement of the leading edge of the liquid sample.

At the end of the analysis chamber, illustrated in FIG. 4, the sample runs into a narrow channel 11, which ends in a hydrophobic stop. The hydrophobic stop is comprised of a very narrow channel 12, preferably having a width of about 50 m, more preferably having a width of about 75 m, most preferably having a width of about 100 m. The length of the channel 12 is preferably about 0.5 mm long, more preferably about 0.75 mm long, and most preferred at least about 1 mm long. This then connects into a wide chamber 13 which is at least 0.5 mm wide, more preferably 0.75 mm wide and most preferably 1 mm wide resulting in a very sharp change in channel width. This design feature has two functions depending on the mode of delivery of the sample to the substrate chamber. In capillary mode of action the resulting sharp change in capillarity acts as a capillary stop, but in pressure driven operations the structure serves another purpose. The very narrow channel 12 causes a pressure drop that restricts the liquid flow velocity and this reduces the dependence of the user-applied pressure.

Thus the novel design of the above-described features are superior to conventional bioarray platforms, as the liquid velocity is regulated and remains the same for similar samples thereby improving the reproducibility and reliability of results.

From the chamber 13 the liquid runs through another narrow channel 14 into a connection channel 15 and further to a circular structure 16, 17 designed to ensure good connection with the fleece material in the waste chamber below.

Air expelled from the analysis area upon loading with a sample or buffer also travels through the waste chamber and is expelled through a vent. Any air bubbles trapped in the analysis area may be forced out by applying pressure through the sample port.

In another preferred embodiment, a slide article, preferably rectangular and plastic, provides for a closed substrate platform with a “straight canal”. The design of the analysis platform allows for liquid sample, injected through a narrow inlet port connected to a narrow tube 730. The inlet port is comprised of an adapter 710, which fits into the inlet port 720, and is conical in shape. The top part of the adapter is constructed to receive a pipette or a syringe needle. The adapter is comprised of rubber or a silicon-based material so that there is a tight contact between the pipette tip or syringe needle when fluid is delivered. The fluid travels from the adapter to the analysis area through a canal which is interrupted by a flow restrictor 740. The flow restrictor serves the purpose of allowing for the liquid sample to be distributed evenly through the analysis area when pressure is applied by a user or automated system. The analysis area is preferably a straight and narrow canal with no turns 750. At a defined area, probes are attached to the bottom of the canal. The fluid travels from the analysis area through a canal 760 and arrives at a buffer chamber 770. The buffer chamber also serves the purpose of relieving any pressure build up of that may be a consequence of the type of assay being performed, e.g. PCR. During the thermal cycles of a PCR assay, steam is produced resulting in a pressure build-up. Connected to the buffer chamber is a short capillary canal that opens into a meandering design waste area 780. The waste area ends in a vent, shaped as a capillary chamber 790.

In another preferred embodiment, the slide is comprised of a bottom surface plastic structure as described above. The top surface of the slide is comprised of a thin plastic film or laminate which is placed over the bottom part of the slide and sealed using heat or adhesive followed by physical pressure to ensure airtight sealing and prevent any liquid or gas from escaping through the seal.

This has the advantage of providing for a thin slide that can be used in many applications or instruments which normally employ a standard microscope slide. The slide is preferably about 1.3 mm thick, more preferably about 1 mm thick, most preferably at least 0.5 mm thick. Another major advantage of the slide is that the thin design, for example 1 mm, is of benefit as many standard confocal scanners have a fixed focus at this distance, whereas with thicker slides, the focus distance can be fine tuned only within a distance of +/−100 m.

Preferred closed substrate platforms of the invention comprise:

a slide element enclosed within a container, preferably a sealed container with an opening that can provide fluid communication to the system, and the slide element preferably being plastic,

wherein the slide comprises a defined area for sample analysis,

wherein the container comprises at least one inlet port for introduction of liquid into the sealed container which delivers liquid to the area for sample analysis preferably via a tube or channel,

wherein the sample analysis area is of a meandering design,

wherein the sealed container comprises at least one vent for expulsion of air from the sealed container,

wherein the sealed container comprises an outlet port for removal of liquid from the area for sample analysis preferably via a tube, channel, or the like, and

preferably wherein the outlet port connects to a waste area located within the sealed container.

Particularly preferred closed substrate platform of the invention comprise:

a slide element enclosed within a container, preferably a sealed container with an opening that can provide fluid communication to the system, and the slide element preferably being plastic,

wherein the slide comprises a defined area for sample analysis,

wherein the sample analysis area is of a meandering design,

wherein the container comprises a first inlet port for introduction of a sample into the sealed container and which delivers the sample to the area for sample analysis preferably via a tube or channel,

wherein the container comprises a first vent for expulsion of air from the sealed container due to introduction of sample through the first inlet port,

wherein the sealed container comprises a second inlet port for introduction of a fluid such as a wash buffer into the container and which delivers the fluid (preferably wash buffer) to the area for sample analysis preferably via a tube or channel, and

wherein the container comprises a second vent for expulsion of air from the container due to introduction of fluid through the second inlet port.

Another preferred closed substrate platforms of the invention comprise:

a slide element enclosed within a container, preferably a sealed container with an opening that can provide fluid communication to the system, and the slide element preferably being plastic,

wherein the slide comprises a defined area for sample analysis,

wherein the container comprises at least one inlet port for introduction of liquid into the sealed container which delivers liquid to the area for sample analysis preferably via a tube or channel,

wherein the sample analysis area is a straight channel,

wherein the sealed container comprises at least one vent for expulsion of air from the sealed container,

wherein the sealed container comprises an outlet port for removal of liquid from the area for sample analysis preferably via a tube, channel, or the like, and

preferably wherein the outlet port connects to a waste area located within the sealed container.

Particularly preferred closed substrate platform of the invention comprise:

a slide element enclosed within a container, preferably a sealed container with an opening that can provide fluid communication to the system, and the slide element preferably being plastic,

wherein the slide comprises a defined area for sample analysis,

wherein the sample analysis area is a straight channel,

wherein the container comprises a first inlet port for introduction of a sample into the sealed container and which delivers the sample to the area for sample analysis preferably via a tube or channel,

wherein the container comprises a first vent for expulsion of air from the sealed container due to introduction of sample through the first inlet port,

wherein the sealed container comprises a second inlet port for introduction of a fluid such as a wash buffer into the container and which delivers the fluid (preferably wash buffer) to the area for sample analysis preferably via a tube or channel, and

wherein the container comprises a second vent for expulsion of air from the container due to introduction of fluid through the second inlet port.

In a most preferred closed substrate platform of the invention is comprised of:

a slide element enclosed within a container, preferably a sealed container with an opening that can provide fluid communication to the system, and the slide element preferably being plastic,

wherein the slide comprises a defined area for sample analysis,

wherein the sample analysis area is a straight channel,

wherein the container comprises an inlet port that is fitted with a rubber or silicon-based adaptor for introduction of a sample into the sealed container,

wherein the adaptor is conically shaped and which allows for introduction of the sample into the adaptor with a minimized risk of spillage or back flow,

wherein the fluid travels from the adapter to the analysis area through a canal which is interrupted by a flow restrictor, afterwhich,

the fluid travels from the analysis area through a canal and arrives at a buffer chamber which is connected to a short capillary canal that opens into a meandering design waste area,

wherein, the waste area ends in a vent, shaped as a capillary chamber.

The closed substrate platform can be comprised of at least one individual straight narrow channel to about five individual straight narrow channels.

As used herein, the term “individual straight narrow channels”, refers to the shape of substrate analysis area and each analysis area is completely separate from the next channel with its own inlet and outlet ports and there is no cross flow of sample between the individual chambers. Each chamber is therefore, a separate structure.

The closed substrate platforms are suitably used in an array format, i.e. where multiple parameters are to be analyzed substantially simultaneously on the substrate platform. As referred to herein, the term “array” indicates a plurality of analytical data points that can be identified and address by their location in two or three-dimensional space, where i.e. identification can be established by the data point physical address.

Typically, the analysis systems of the invention utilize test samples that are in fluid form. For instance, test samples derived from humans or other mammals, or plant sample, may originate from blood, urine, or solid tissue or cells and will suitably be pre-treated to enrich or dilute the material to provide an optimized test sample.

In preferred analysis systems of the invention, the system will hold an accurate and reproducible volume of test sample fluid, e.g. a volume of about 5 μl to about 10 μl is preferred, although other volumes also can be employed if desired. Sample may be added at volumes of up to at least about 100 μl.

In a further preferred embodiment of the closed substrate platform is that it is designed to operate with very small volumes of sample and buffer. The overall thickness of the substrate platform is preferably at least about 1 mm in order to make it as compatible with existing equipment designed for handling microscope slides as possible. This design is advantageous for applications wherein a limited number of spots, for example, at least about 300 spots, more preferably at least about 700 spots, most preferably at least about 1000 spots, need to be analyzed. The amount of target or sample solution required to perform the analysis is at least about 5 μl to at least about 100 μl. A preferred volume is at least about 1 μL, more preferably the volume of sample is at least about 5 μL , most preferably the volume of sample is at least about 7 μL.

According to the invention, the sample or buffer is loaded by inserting a pipette in the sample port, which has the shape of a hole connecting the inlet chamber to the outside of the substrate platform. The inlet chamber is long and narrow to ensure that liquid injected into the chamber is in contact with all walls of the chamber and thus pushing any existing bubbles forward to the end of the chamber and out through a small channel connecting the inlet chamber to the analysis chamber. The diameter of the connecting channel is at least about 100 μm , more preferably the diameter of the connecting channel is at least about 250 μm, most preferred the diameter of the connecting channels is at least about 500 μm.

On its way to the analysis chamber the liquid sample passes through a “pressure reducer” in the shape of a hydrophobic stop. The channel narrows down from about 500 μm to about 100 μm for a length of at least about 1 mm and then expands to a chamber. The chamber is at least about 1 mm wide and at least about 2 mm long. At the opposite end of the chamber the liquid exits through another channel with a diameter of at least about 100 μm and into a larger channel with a diameter of at least about 500 μm. The lengths described here are illustrative and are not meant to be restrictive. Various dimensions can be used according to the physical and chemical properties of the liquid used, for example, viscosity, hydrophobicity, hydrophilicity, and the like.

From the above channel, the sample or buffer runs to the analysis chamber, which is at least about 2 mm wide, at least about 20 mm long and at least about 75 μm deep, having a total volume of at least about 3 μL. For example, if a spotting pitch of 200 μm is used this chamber will be able to contain 600 spots. The lengths described here are illustrative and are not meant to be restrictive. Various dimensions can be used according to the physical and chemical properties of the liquid used, for example, viscosity, hydrophobicity, hydrophilicity, and the like.

When the sample or buffer exits the analysis chamber it runs through another hydrophobic stop before entering the waste chamber.

To ensure the rigidity of the substrate platform (where most of the internal volume is taken up by the waste chamber and thus not contributing to the rigidity) a number of supporting walls are placed in the waste chamber, giving the chamber the shape of a meander. The waste chamber may be separated from the analysis area by a narrow channel. This is to avoid diffusion of washed out hybridization components from the waste chamber into the analysis area.

The bottom of the slide is indented directly beneath the analysis chamber for easy access with a peltier element or other heating/cooling device to control the temperature inside the chamber. Preferably the material thickness of the polymer that comprises the bottom portion of the slide is locally very thin, for example, preferred thickness is at least about 50 m, more preferably the thickness is at least about 75 μm, most preferred thickness is at least about 100 μm, in the area directly beneath the chamber to ensure good heat transfer from/to the heating/cooling device. The thickness of the material may vary depending on the material used to construct the slide, wherein each material has different heat transfer properties. A selection of materials for construction of the slide is described infra.

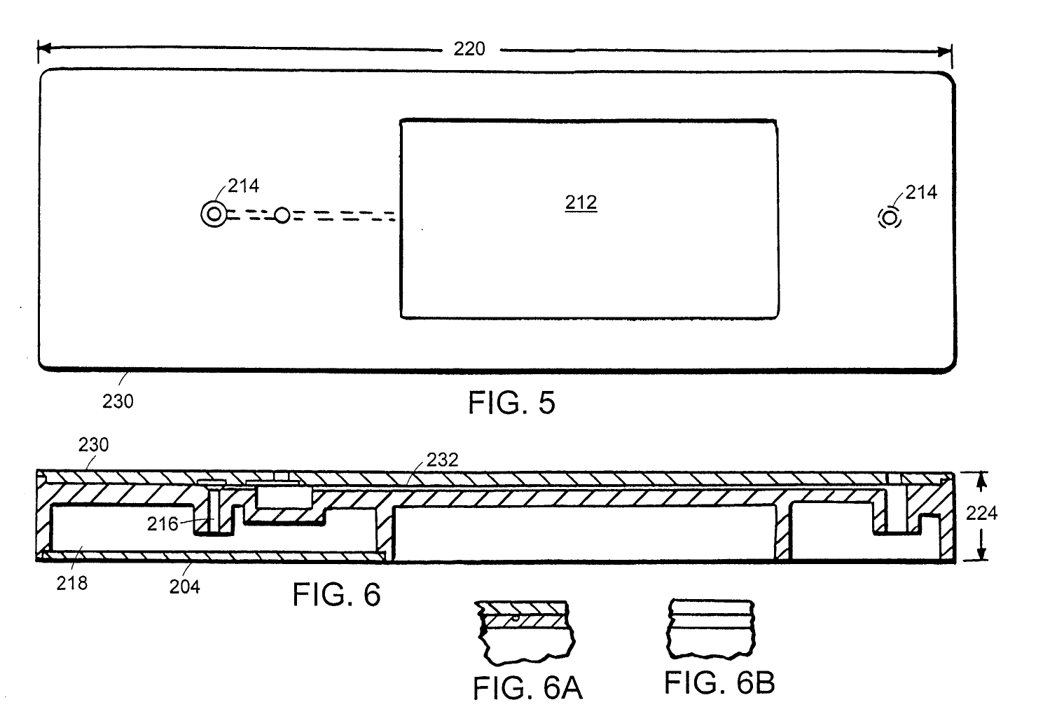

The invention also provides a closed slide with the general dimensions of a standard microscope slide as shown in FIGS. 5 and 6. The closed slide contains the substrate platform enclosed within a sealed container. The closed slide further contains an integrated microfluidics system to permit sample loading, manipulation, washing, etc. The closed substrate platform may be constructed as a single complete unit wherein the substrate platform and the container are constructed as a single unit. Alternatively, the substrate platform may be constructed separately from the container and later assembled into a single unit. The substrate platform need not be made of the same material as the container. The substrate platform in the closed slide may be constructed from any polymer which contains an acceptable level of intrinsic background fluorescence. Other suitable materials of constructions of analysis systems of the invention, including metals where analysis methods would include detecting electric signal or where a metal layer (e.g. gold) is deposited for mass spectrum analysis or other purposes. The closed substrate platforms are suitably used in an array format, i.e. where multiple test samples are analyzed substantially simultaneously on the substrate platform. As referred to herein, the term “array” indicates a plurality of analytical data points that can be identified and address by their location in two or three-dimensional space, where i.e. identify can be established by the data point physical address.

Typically, the analysis systems of the invention utilize test samples that are in fluid form. For instance, test samples derived from humans or other mammals, or plant sample, may originate from blood, urine, or solid tissue or cells and will suitably be pre-treated to enrich or dilute the material to provide an optimized test sample.

Preferred closed analysis systems of the invention are sealed cartridges wherein an analytical area are housed within the cartridge. The structure can contain multiple inlet ports, typically two inlet ports, one for sample introduction, and one for washing solutions. The inlet ports can be operated by a variety of methods, e.g. standard pipettes, either manually or by robot. The inlet port provides introduction of the test sample into an analytical area, suitably holding the same preferred volumes of test sample fluid as discussed immediately above. The system will hold an accurate and reproducible volume of test sample fluid. A volume of from about 1 μl to about 10 μl is particularly preferred, where the introduction of a total of about 5 μl to about 30 μl will fill the microfluidic channels and analytical cavity. Optionally, a waste chamber can be integrated into the closed system to retain all added fluids including excess sample, washing buffers and other reagents. The overall dimensions of the closed system suitably may correspond to about 20 to about 30 mm wide, from about 70 to about 80 mm long and from about 0.1 to about 6 mm thick. A most preferred thickness of the slide is from about 0.6 mm to about 1 mm. Preferably, the closed system does not contain any moving parts or pumps. Movement of fluid through the system can be suitably accomplished by capillary forces and/or pressure introduced from outside the system such as during fluid introduction. The closed system should have an outlet for escape of air as fluid passes into and through the system. The closed system suitably can be compatible with a microplate format, wherein a holder that has the same outer dimensions as a standard microplate will hold multiple closed systems of the invention, typically four closed systems.

In another preferred embodiment, the slide is comprised of a bottom surface plastic structure as described above. The top surface of the slide is comprised of a thin plastic film or laminate which is placed over the bottom part of the slide and sealed using heat or adhesive followed by physical pressure to ensure airtight sealing and prevent any liquid or gas from escaping through the seal.

This has the advantage of providing for a thin slide that can be used in many applications or instruments which normally employ a standard microscope slide. The slide is preferably about 1.3 mm thick, more preferably about 1 mm thick, most preferably at least 0.5 mm thick. Another major advantage of the slide is that the thin design, for example 1 mm, is of benefit as many standard confocal scanners have a fixed focus at this distance, whereas with thicker slides, the focus distance can be fine tuned only within a distance of +/−100 m.

The closed slide preferably contains a clear window within the top of the closed container corresponding the analysis area. The window allows the user to monitor liquid flow into and out of the analysis area and determine whether air bubbles are present. The window further allows biomolecules bound to the analysis area to be detected by a scanner, reader, etc. without opening the sealed container.

The closed slide preferably contains finger holds in the form of ridges or indents on the sides of the closed container. The finger holds are preferably paired on opposite sides of the closed container. The closed slide may also contain other surface contours such as recessed or raised regions which may perform functional or decorative purposes.

The casing of the closed slide may be constructed of a variety of materials, such as polycarbonate and the like. All or part of the casing of the closed slide may be transparent, opaque, frosted, etc. Additionally, all or part of the casing may be any one of or a variety of colors and may contain surface markings such as numbers, words, pictures, company logos, bar codes, etc. In a particularly preferred embodiment, the casing may contain labels for the inlet and outlet ports to indicate, for example, where sample and wash materials may be introduced and waste or air may be expelled (i.e. vents), etc.

The closed slide is preferably dimensioned so as to fit into any instrument or device which is capable of receiving a standard microscope slide. Specifically, the closed slide is preferably from about 20 to about 30 mm wide 222, from about 70 to about 80 mm long 220 and from about 1 to about 10 mm thick 224. More specifically, the closed slide is preferably about 25 mm wide by 76 mm long by 1 mm thick.

The closed slide preferably contains inlet ports 214 for sample loading, buffer washing and air expulsion upon washing or loading. The inlet ports may be arranged in a variety of configurations so as to allow sample loading and washing without contamination of the analysis area. The sample ports are preferably funnel shaped with the wide end of the funnel toward the outside of the casing and the narrow end toward the inside of the casing, in order to facilitate introduction of liquid into the closed slide. Preferably the sample and buffer ports may be configured so as to receive liquid from a variety of sources such as a pipette tip, a syringe, a tube or channel, a robotics system, etc. In a particularly preferred embodiment, the ports are configured so as to be capable of receiving liquid from a standard pipette tip.

The sample ports preferably contain a septum (i.e. a partition or dividing wall) which serves as a self-closing inlet to prevent contamination. The septum preferably will open upon contact with a pipette tip, or other instrument used to introduce liquid into the slide, and will close or reseal upon removal of the pipette tip or other such instrument. The septum is preferably constructed of a sealable material such as, for example elastomer, silicone rubber, teflon, etc. As used herein, the term “sealable” means that after introduction of sample, the septum will be able to close and maintain a closed or sealed environment without introduction of unwanted air, liquid, etc. from the outside and without substantial loss of air, fluid, etc. from the inside.

The sample or inlet ports may also be fitted with a rubber adaptor, or silicon-based material, which is conical in shape and fits tightly into the inlet ports. The adapter fits, at one end into an inlet at the bottom face of the slide and the top end is adapted to receive a pipette or syringe tip.

In a most preferred embodiment, the waste chamber is of meandering design, ending in a vent shaped as a capillary canal. The waste area does not contain any fleece or other material but the force used when the sample is introduced into the inlet or sample port is the force that drives the sample through the microfluidic device and into the waste area.

The closed slide can also, preferably contain port 216 and a waste area 218 located within the sealed container beneath the substrate platform. The waste area is preferably configured so as to be able to receive at least about 1 ml of fluid and up to about 5 ml of fluid from the analysis area. In a preferred embodiment the waste area contains an absorbent material such as a gel, cloth, fleece. etc. which is capable of soaking up the waste fluid and preventing any backflow of the waste material into the analysis area. The sorbent material is then able to work as a capillary pump, enabling the drawing of the liquid out of the fluidic structures and into the waste chamber, driven by the very high capillary force of the fleece.

For optimal coupling of the fluidic system to the fleece a special design element has been developed for controlled and continuous flow of the liquid into the waste chamber. The inlet into the waste chamber 216 consists of a neck with notch-structured zones, preferably star shaped, of the waste inlet connectably coupled to the waste chamber, preferably to the absorbing material inside the waste chamber of the fluidic device. The notches are the coupling element which thereby cause increased contact surface between the neck and the fleece. The wedge-shaped notches cause an initial sucking force due to capillary forces. See European patent application serial no. EP 1 013 341 A2.

In the meandering design or straight channel design substrate analysis platform, the waste area may not contain fleece. The pressure applied when introducing the sample into the inlet port is sufficient to drive the sample through the microfluidic structure of the slide element.

The closed slide preferably contains inlet ports 1, FIG. 2, for sample loading, buffer washing and air expulsion upon washing or loading. The inlet ports may be arranged in a variety of configurations so as to allow sample loading and washing without contamination of the analysis area. The sample ports are preferably funnel shaped or conical shaped with the wide end of the funnel toward the outside of the casing and the narrow end toward the inside of the casing, in order to facilitate introduction of liquid into the closed slide. Preferably the sample and buffer ports may be configured so as to receive liquid from a variety of sources such as a pipette tip, a syringe, a tube or channel, a robotics system, etc. In a particularly preferred embodiment, the ports are configured so as to be capable of receiving liquid from a standard pipette tip.

The sample ports preferably contain a septum (i.e. a partition or dividing wall) which serves as a self-closing inlet to prevent contamination. The septum preferably will open upon contact with a pipette tip, or other instrument used to introduce liquid into the slide, and will close or reseal upon removal of the pipette tip or other such instrument. The septum is preferably constructed of a sealable material such as, for example elastomer, silicone rubber, teflon, etc. As used herein, the term “sealable” means that after introduction of sample, the septum will be able to close and maintain a closed or sealed environment without introduction of unwanted air, liquid, etc. from the outside and without substantial loss of air, fluid, etc. from the inside.

The closed slide preferably contains channels 14 and 15, (FIG. 4) leading to a circular structure 16 and 17 designed to ensure a good connection with a waste area 218, FIG. 6, located within the sealed container beneath the substrate platform. The waste area is preferably configured so as to be able to receive at least about 0.1 ml of fluid from the analysis area. In a preferred embodiment the waste area contains an absorbent material such as a gel, cloth, fleece. etc. which is capable of soaking up the waste fluid and preventing any backflow of the waste material into the analysis area. The sorbent material is then able to work as a capillary pump, enabling the drawing of the liquid out of the fluidic structures and into the waste chamber, driven by the very high capillary force of the fleece.

For optimal coupling of the fluidic system to the fleece a special design element has been developed for controlled and continuous flow of the liquid into the waste chamber. The inlet into the waste chamber 216, FIG. 6, comprises a neck with notch-structured zones 16+17 FIG. 4, preferably star shaped, of the waste inlet connectably coupled to the waste chamber, preferably to the absorbing material inside the waste chamber of the fluidic device. The notches are the coupling element which thereby cause increased contact surface between the neck and the fleece. The wedge-shaped notches cause an initial sucking force due to capillary forces. See European patent application serial no. EP 1 013 341 A2.

In fluidic devices, particularly in use with miniaturized analysis such as with bioarrays, there is the necessity to spread a fluid stream homogeneously from a narrow channel into a wide area. Often times it is necessary to disperse fluid between structures with very different cross-sections, for example, between an incoming channel and a hybridization area or a reaction chamber. Preferred analysis systems of the invention include, “meandering”, “butterfly” and “cascade” structures of channels to contend with such difficulties. A most preferred analysis system, according to the present invention is the meandering structure or an analysis area comprising just a straight narrow channel.

The meandering, butterfly and cascade channel systems of devices of the invention can provide:

a) uniform spreading of the fluid into a homogenous film;

b) homogenous wetting of the surface in a reaction chamber (for example in a hybridization chamber);

c) entrance of the fluid into an analysis and/or reaction and/or detection and/or indication area with a homogenous flow profile between two plates (the lid and the base of the substrate platform); and

d) uniform narrowing (reunion) of the liquid afterwards (after the analysis area).

The analysis chamber is most preferably a channel meandering up and down the slide. The pressure applied by the user causes the sample or buffer to distribute evenly through the analysis area. In the 180° turns of the analysis area 10 (see FIG. 3) the path taken by the liquid at the outer part of the curve is longer than the path taken by the liquid at the inside of the turn. Therefore the outer liquid needs to travel faster than the inner liquid to obtain an even liquid front velocity across the width of the channel. This is implemented by reducing the width of the meandering channel from at least about 3 mm to at least about 2 mm in the turns, thus reducing the differences in travel distances and therefore the differences in flow velocities. The widths of the meandering channel compared to the widths of the turns can be varied as long as the liquid front-velocity remains even.

The butterfly structure comprises a symmetrical “delta”-structured channel system of bifurcations with a constant value of the cross-section (decreasing channel depth and increasing channel width with increasing number of bifurcations). The butterfly channel system initiates and/or terminates with a V-shaped border line on the wide end of the tree structure.

A constant cross-section guarantees a constant flow rate as well as increasing capillary force. A V-shaped front line assists in eliminating a smiling effect and non-uniform channel depths enable dispersement of fluid to a homogenous film, thus achieving a homogeneous flow of the liquid into the analysis area and diminishing a smiling-effect which causes an opposite flow profile by the V-shape (“anti-smiling-tree”). The peak in the middle of the V-shaped front line may be sharp or rounded. In addition to use in filling an area such as an analysis area, the butterfly structure can alternatively be used to narrow the fluid stream.

Preferred analysis systems of the invention may also comprise an additional channel system (cascade) of a similar structure where a triangular shaped structure with steps (terraces) of increasing depth in the direction to the top of the triangle (decreasing capillary force) enables the homogenous spreading or narrowing of the fluid stream. The cascade consists of at least of two areas with different depths and therefore with different capillarities (different capillary forces). As a result, flowing fluid fills out each step completely before it climbs up or down to the next terrace. The edges of cascaded terraces may include notches as described herein for the inlet to the waste chamber, for an easier wetting of the following terrace.

Preferred fluid transfer devices of the invention may include a pre-shooter stop to combat the difficulties associated with spreading fluid in fluidic microdevices. If a fluid enters into a wide but very narrow area between two plates; for example, between the lid and the base of a fluidic device, the liquid tends to flow at the edges of the area faster than in the middle due to regions of higher capillary force in the corner of the edge. A “pre-shooter” results if the liquid shoots very quickly along an edge. In addition, a “smiling effect” results, which means that the front line of a flowing fluid for example in an analysis area is not homogeneous and lacks a steady front, which is instead curved like a smiling face.

Preferred devices of the invention include one or more pre shooter stops, which can avoid the occurrence of undesired “pre-shooters” and provide a homogeneous fluid front line. Pre-shooter stops are irregular shaped structures, preferably triangular or saw tooth shaped structures, placed at strategic points, thus avoiding pre-shooters at the borders of wide, flat areas (for example, in the analysis area) and achieving a homogeneous liquid flow into and through this area. The structures disturb the capillary force along the edge via discontinuation. It is possible to place only one pre-shooter-stop on critical positions (for example, on each side on the border between the end of the “butterfly” structure and the beginning of the hybridization chamber). In addition it is also possible to place more than one “pre-shooter-stop” along the border of an area (for example, the analysis area). The functionality of the pre-shooter-stops depends on the angle and the height of the tooth, because the greater the height of the stop, the more disruption results.

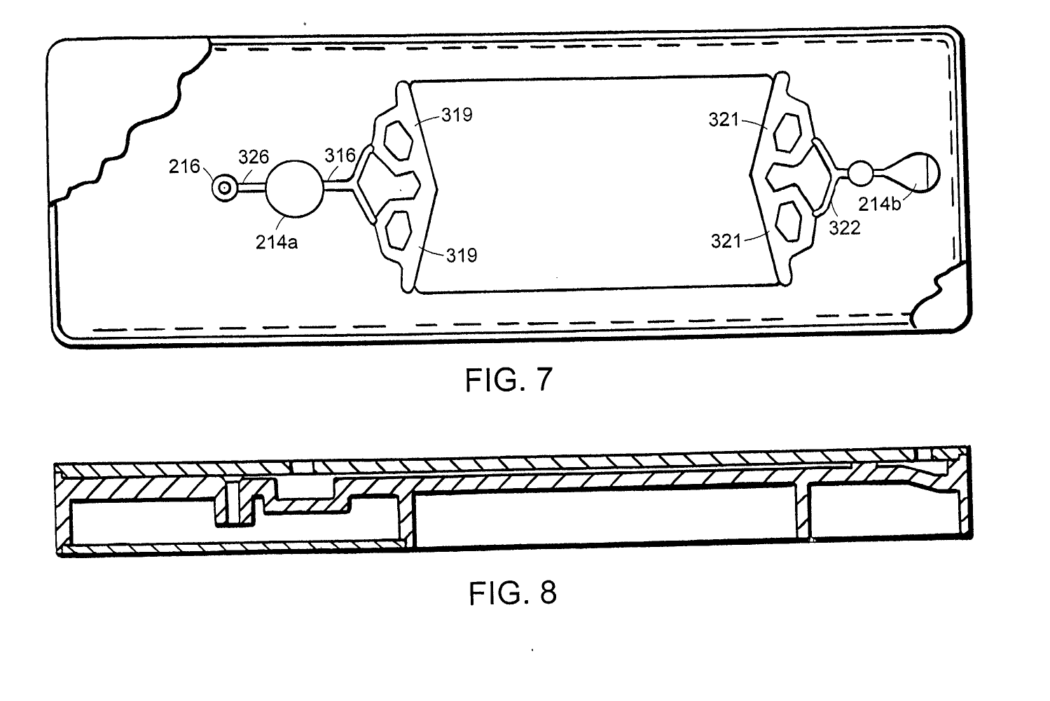

Another preferred embodiment of the closed slide substrate platform 300 is shown in FIGS. 7 and 10. The top cover of the closed slide 230 contains inlet ports for sample loading 214a and buffer washing 214b. The sample and buffer ports are preferably located on opposite lengthwise sides of the analysis area 212. A sample loaded through the sample port 214a travels through a butterfly system, through a Y shaped tube 316 to a V shaped outlet port 318 which expels liquid into the cavity above the analysis area 212 through 319 located toward the outside edges of the analysis area. The loaded sample then fills the analysis area cavity by capillary action starting at the outside edges and moving across and in until the entire cavity is filled with fluid and no air bubbles remain. The hydrophobicity/hydrophilicity of the surface of the substrate in the analysis area may be optimized so as to achieve efficient spreading of the sample across the analysis area.

Additional structures of the device may also be used to achieve efficient entry and spreading of fluids in the device. To fill a fluidic structure with fluid, a “capillary driven sample inlet chamber” is advantageous. This chamber is able to initially hold fluid which is pipetted into the device, in the inlet port 214. From this chamber the fluidic channels in the device require continuous filling with liquid to a required extent in order to maintain capillary action. This has been solved by using a sample inlet chamber which comprises at least one vertical wedge-shaped capillary notch which extends from the bottom of the chamber to its top, thus enabling the continuous filling of the channels of the fluidic device as well as the analysis area with the fluid. The content of the chamber fills the channel system, driven by the capillary force of the vertical notch.

Such a chamber can be essential for the above mentioned embodiment of a fluidic device in which the transport of a liquid from the inlet port to the outlet port through the analysis substrate occurs using capillary forces as driving forces.

A lengthwise cross-sectional view of the closed slide is shown in FIG. 6. The substrate platform 210 is located just inside the top cover 230 of the closed container. As shown in FIG. 56, the inlet ports 214 allow fluid to be introduced into the closed slide and pass fluid from the port to the analysis area 212 via small tubes or channels 232. The tubes or channels 232 open into the analysis area 212 which is separated from the top cover 230 of the closed container by a defined amount thus forming a cavity capable of containing liquid of a specific volume over the analysis area of the substrate platform. Liquid is removed from the analysis area by traveling down one of the tubes or channels 232 to the exit port 216 which connects the tube or channel to the waste area 218.

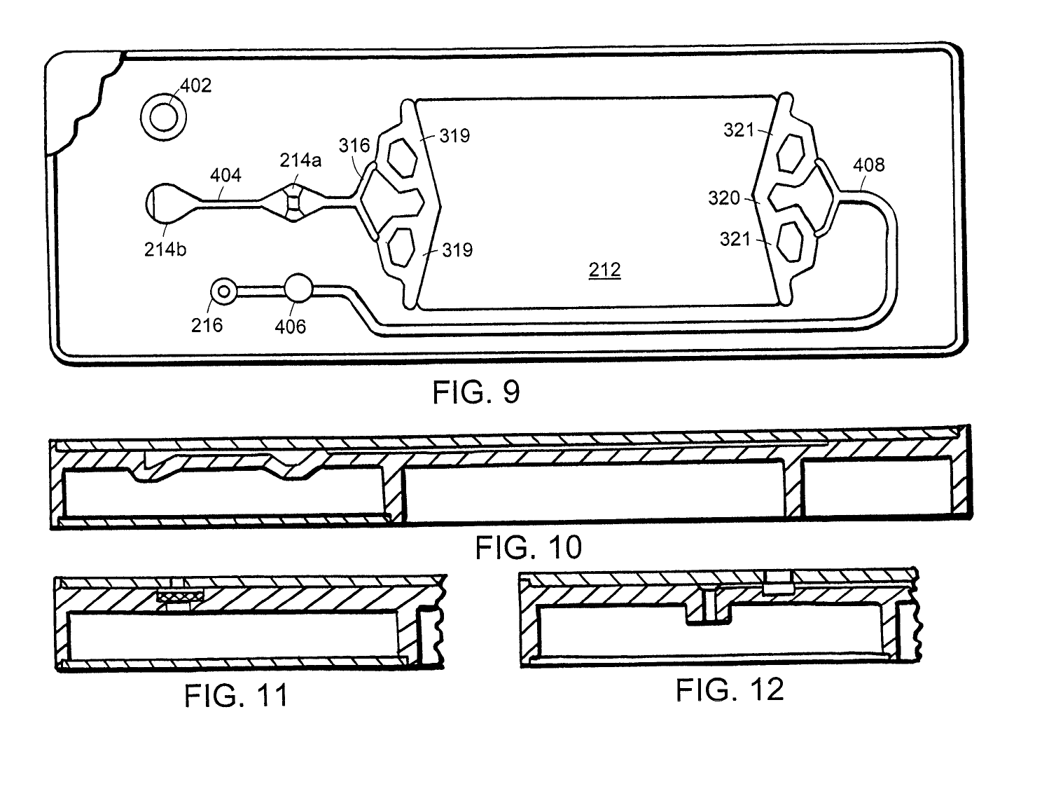

A preferred embodiment of the closed slide substrate platform 300 is shown in FIG. 9. The top cover of the closed slide 230 contains inlet ports for sample loading 214a and buffer washing 214b. The sample and buffer ports are preferably located on opposite lengthwise sides of the analysis area 212. A sample loaded through the sample port 214a travels through a Y shaped tube or channel 316 to a V shaped outlet port 318 which expels liquid into the cavity above the analysis area 212. The loaded sample then fills the analysis area cavity by capillary action starting at the outside edges and moving across and in until the entire cavity is filled with fluid and no air bubbles remain. The hydrophobicity/hydrophilicity of the surface of the substrate in the analysis area may be optimized so as to achieve efficient spreading of the sample across the analysis area.

A most preferred embodiment of the analysis area is a meandering design. The analysis area may comprise at least two separate inlet ports wherein each inlet port is connected to a separate meandering analysis chambers. In this way multiple samples may be analyzed at the same time under the same conditions, using the same slide. This is especially important wherein a user may need to compare different samples under the same experimental conditions. The analysis chambers may be comprised of similar molecules when different samples are being tested or the analysis chamber may comprise different molecules when the same sample is to be tested against a different molecules. This allows for uniform experimental conditions, reducing the mean error from experiment to experiment. Each separate subchamber preferably contains its own separate microfluidics system including inlet ports, outlet ports, vents, tubes or channels, etc.

In another preferred embodiment of the analysis area is a straight channel design leading from the inlet port and ending with the outlet port. The closed substrate platform may be comprised of at least about one straight channel to at least about five straight channels, each substrate platform as a separate entity with its own inlet and outlet ports so that there is no cross-contamination of samples

Alternatively, the analysis area may contain one or more extended channels, including an extended channel that traverses repeatedly through the analysis area.

In systems having multiple flow channels, those flow channels may each have separate microfluidic systems (e.g. inlet and outlet ports, waste chambers), or the two or more channels may share a single microfluidic system.

In general, regardless of the design of the analysis area, the analysis area may be washed by introducing buffer through a buffer port. FIG. 5 shows a rectangular analysis area and buffer is introduced via the buffer port 214b. Fluid loaded through the buffer port 214b travels through another Y shaped tube or channel 322 to another V shaped outlet port 320 which expels liquid into the cavity above the analysis area 212 through two holes 321 located toward the outside edges of the analysis area. The buffer fills the analysis area cavity via pressure applied from the instrument used to load the buffer.

Air expelled from the analysis area upon addition of buffer to the closed container is expelled through a vent 402 which opens from the waste area 218 to outside the sealed container.

Vent 402 is an outlet structure which is sealed (closed) preferably with a hydrophobic, air permeable material. That structure will allow for escape of air, while maintaining fluid in the device. The vent may be constructed of any porous hydrophobic polymer fit or more preferably a polymer membrane. Such a structure enables the expulsion of air out of the device, the degassing of the fluid within the device, and a hydrophobic stop for the fluid. The vent with hydrophobic cap also may act as a “marker” for the identification of the end of the filling of the substrate platform.

Fluid in the analysis area cavity is expelled by pressure applied from the buffer port 214b. The fluid travels back towards the sample inlet through the V shaped outlet port 318 and Y shaped tube or channel 316. The fluid then passes by the inlet port 214a through a short tube or channel 36. into the waste outlet port 216. The waste outlet port expels the fluid from the analysis area into a waste area 218 which is located within the sealed container beneath the substrate platform 210.

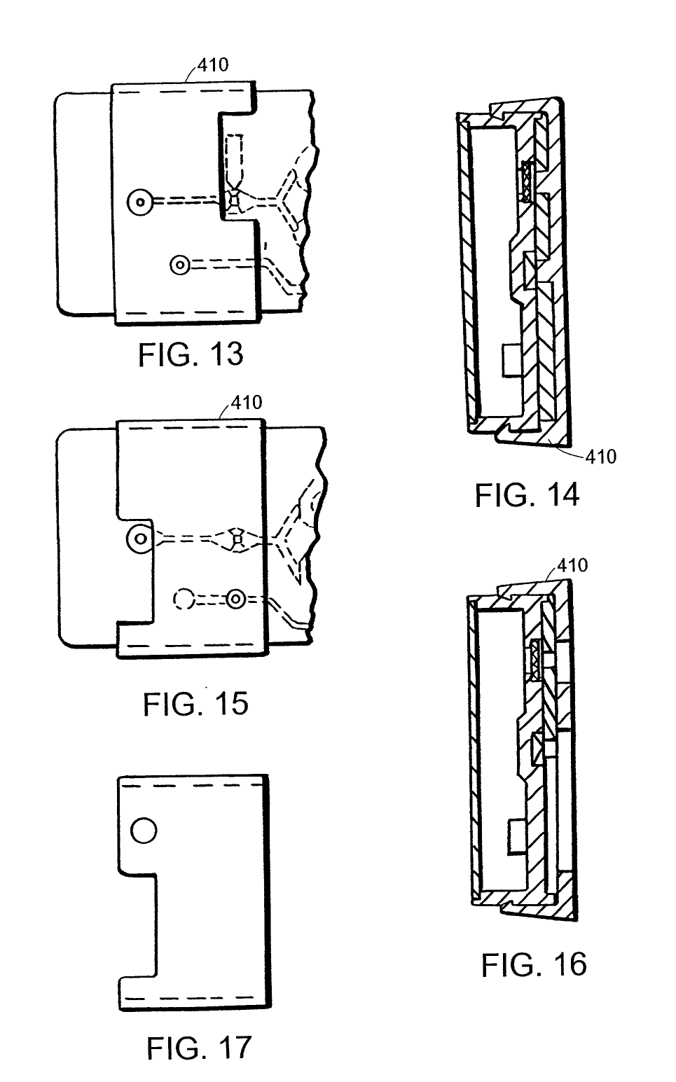

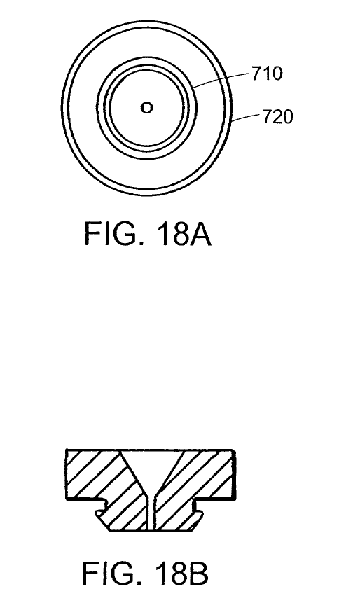

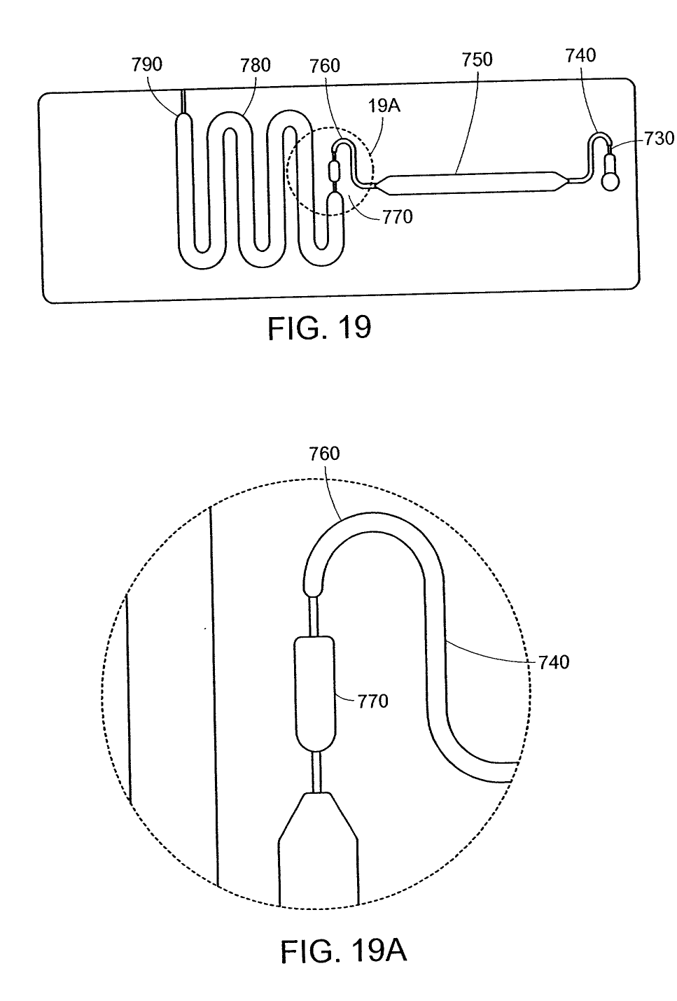

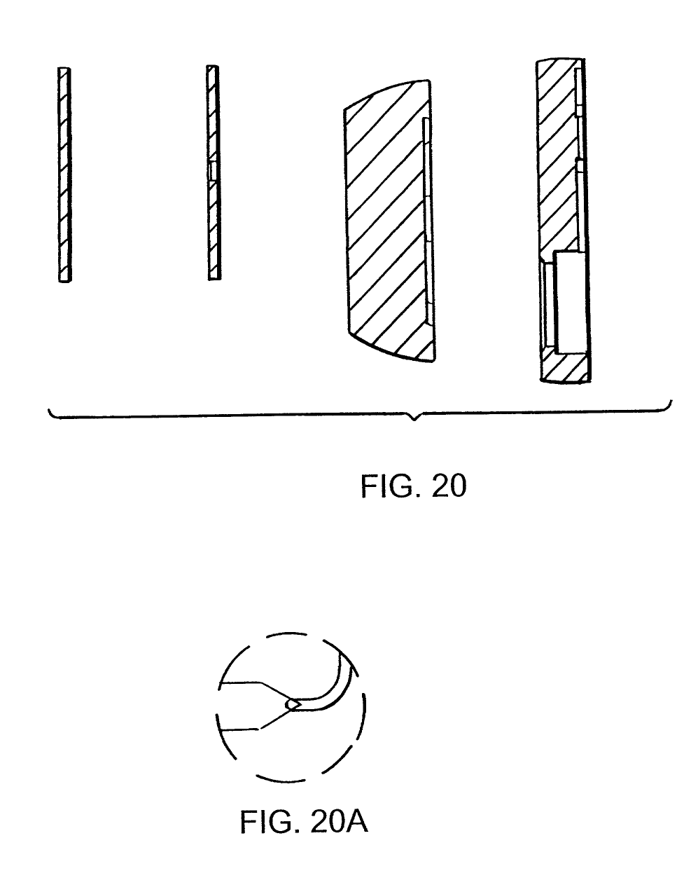

A further embodiment of the closed substrate platform is shown in FIGS. 9-17, 400. In this embodiment, the sample port 214a is located between the buffer port 214b and the analysis area 212. A sample loaded through the sample port 214a travels through a Y shaped tube or channel 316 and a V shaped outlet port 318 and is expelled into the cavity above the analysis area 212. The loaded sample fills the analysis area via pressure applied from the loading instrument (i.e. pipette, syringe, etc.).

Air expelled from the analysis area upon loading with a sample or buffer wash is expelled through a vent 406. Any air bubbles trapped in the analysis area may be forced out by applying pressure through the sample port 214a.

The analysis area may be washed by introducing buffer through the buffer port 214b. Liquid loaded through the buffer port 214b travels through a short tube or channel 404 into the sample port 214a area and then proceeds into the analysis area 212 via the same Y shaped tube or channel 316 and V shaped outlet 318, 319 as used for sample loading. Air expelled from the analysis area upon addition of buffer to the closed container is expelled through a vent 402 which opens from the waste area 218 to outside the sealed container.

Fluid in the analysis area 212 is expelled by pressure applied from the buffer port 214b. The liquid travels into a V shaped exit port 320 through holes 321 and then into a Y shaped tube or channel 408 The fluid travels through the tube or channel 408 past the air vent 406 to the waste outlet port 216 which expels the fluid from the analysis area into the waste area 218 located beneath the substrate platform 210.

The air vent 406 and waste outlet port 216 need not be located on the same side of the analysis area as the sample 214a and buffer 214b ports.

To contend with such requirements, a “capillary stop” has been developed. At least one capillary stop is necessary to halt the fluid. The stop comprises a transition section of channels with different capillarities. Such an element consists of a gap of low capillarity between two channels with high capillarity. The fluid ceases at the end of the first channel and does not enter into the gap.

A preferred use of capillary stops is a combination of two capillary stops, such as in front of the waste inlet. The first capillary stop halts the liquid during the filling of the device, while the second stop halts the liquid during a method such as a heating step which is necessary for the analysis or assay reaction. Such combination of stops enables to stop the flow before thermal expansion and after thermal expansion of the liquid.

Additional stops may be incorporated at desired sites, such as between the inlet chamber and the washing buffer inlet. This stop avoids the flow of liquid from the filling chamber backwards into the buffer inlet.

Opposing a capillary stop, we have also developed an “anti-stop” structure which enables a split of fluid and continuous flow through bifurcations. Under normal circumstances, splitting of a liquid stream using a T-shaped bifurcation is unreliable because of unavoidable broadening of the channel (it works like a stop). Thus, the fluid halts at the gap of capillary force.

The advantage of the developed “anti-stop” is essentially given by the shape of the bifurcation, the “Y” branches of the channel systems. In contrast to an unsuitable T-shaped bifurcation, the invention provides a curved V-shaped bifurcation where the “top of the V” reaches deep into the entrance. The “top of the V” can be a triangular shaped sharp structure inside the bifurcation. Because the top of the V reaches into the source of the fluid (thus creating a “Y” structure), the capillary force is not broken as in the traditional T bifurcation, and the fluid maintains flow.

As an alternative, or in addition to, the septum, access to the sample port 214a, buffer port 214b and air vents 402 and 406 may be controlled using a ‘cover clip’ 410. The cover clip 410 is a plastic U shaped object which can snap onto the closed slide and cover various combinations of the ports and vents. The cover clip may contain a variety of cutouts for exposure or access of particular ports. Preferably, the cover clip contains an indent for access of an inlet port and a hole for exposure of the corresponding vent. For example, the sample port 214a and air vent 406 may be left exposed while the buffer port 214b and buffer vent 402 are covered. This ensures that sample is correctly loaded into the analysis area 212 and does not escape through the buffer port 214b. The cover clip also serves to ensure that the user injects sample and/or buffer into the correct port and reduces the risk of contamination. The cover clip may also be configured so as to cover all of the ports and vents simultaneously.

The cover clip may be transparent, opaque, frosted, etc. and may be clear, colored, etc. The cover clip may contain raised, etched, indented, etc. regions. The cover clip may also contain markings such as numbers, words, pictures, company logos, bar codes etc. In a particularly preferred embodiment, the cover clip will contain labels to indicate the location of sample ports, wash ports, air vents, etc. to facilitate manipulation of the slide.

In another embodiment, access to the inlet ports and vents may be controlled through the use of stickers adhered to the surface of the casing. The stickers may cover one, several or all of the ports simultaneously. The stickers may be colored or contain markings such as numbers, words, pictures, etc. Preferably, the stickers contain labels to indicate the location of sample ports, wash ports, air vents, etc. to facilitate manipulation of the slide.

The closed substrate configuration which is comprised of a meandering analysis chamber has modifications which enhance the reproducibility of experiments by ensuring even liquid flow, a higher surface to volume analysis area, greater sample to analysis substrate interaction, and decreased risk of air-bubbles. The different substrate analysis platforms described herein have a similar flow through of sample form an inlet port and expulsion of waste through an outlet port.

The most preferred closed substrate platform is of a meander design as shown in FIG. 1 or the straight channel design shown in FIG. 19. According to the invention, the sample or buffer is loaded by inserting a pipette into the sample port, which almost traverses the full thickness of the closed slide as illustrated in FIG. 2E. The sample port is, preferably conical to ensure a tight fit to the tip of the pipette 1. The sample or buffer runs via a small channel 2 to a position directly beneath the inlet chamber and further upwards via a small channel 3 to the bottom end of this chamber 5. The U-shaped inlet causes a pressure drop over the inlet structure from the pipette to the inlet chamber and this reduces the dependence of the user-applied pressure on the velocity of the liquid front in the analysis chamber.

The inlet chamber 5, illustrated in FIG. 2, is long and narrow to ensure that liquid injected into the chamber is in contact with all walls of the chamber and thus pushing any existing bubbles forward to the end of the chamber and out through a small channel 8 connecting the inlet chamber 5 with the analysis chamber 9.

The analysis chamber is most preferably a channel meandering up and down the slide. The pressure applied by the user causes the sample or buffer to distribute evenly through the analysis area. In the 180° turns of the analysis area 10 (see FIG. 3) the path taken by the liquid at the outer part of the curve is longer than the path taken by the liquid at the inside of the turn. Therefore the outer liquid needs to travel faster than the inner liquid to obtain an even liquid front velocity across the width of the channel. This is implemented by reducing the width of the meandering channel from at least about 3 mm to at least about 2 mm in the turns, thus reducing the differences in travel distances and therefore the differences in flow velocities. The widths of the meandering channel compared to the widths of the turns can be varied as long as the liquid front-velocity remains even.

As used herein, “even liquid front velocity” refers to the even rate of movement of the leading edge of the liquid sample.

At the end of the analysis chamber, illustrated in FIG. 4, the sample runs into a narrow channel 11, which ends in a hydrophobic stop. The hydrophobic stop is comprised of a very narrow channel, preferably having a diameter of about 50 :m, more preferably having a diameter of about 75 :m, most preferably having a diameter of about 100 μm. The length of the channel 12 is preferably about 0.5 mm long, more preferably about 0.75 mm long, and most preferred at least about 1 mm long. This then connects into a wide chamber 13 which is at least 0.5 mm wide, more preferably 0.75 mm wide and most preferably 1 mm wide resulting in a very sharp change in channel width. This design feature has two functions depending on the mode of delivery of the sample to the substrate chamber. In capillary mode of action the resulting sharp change in capillarity acts as a capillary stop, but in pressure driven operations the structure serves another purpose. The very narrow channel 12 causes a pressure drop that restricts the liquid flow velocity and this reduces the dependence of the user-applied pressure.

From the chamber 13 the liquid runs through another narrow channel 14 into a connection channel 15 and further to a circular structure 16, 17 designed to ensure good connection with the fleece material in the waste chamber below.

Air expelled from the analysis area upon loading with a sample or buffer also travels through the waste chamber and is expelled through a vent. Any air bubbles trapped in the analysis area may be forced out by applying pressure through the sample port. However, as discussed above the waste area need not contain fleece as the pressure applied by the user or automated source is sufficient to expel any waste material through the waste chamber.

An illustrative example of use of the meandering substrate analysis platform or straight channel design is single nucleotide polymorphism (SNP) analysis. This example is not meant to be restrictive in any way but is illustrative of how the analysis platform is used. A first step provides for the preparation of solutions comprising the desired capture probes. For alignment purposes or a method to detect the location of the capture probes a solution of, for example, a t-15 oligo modified in the 5′-end with anthraquinone and in the 3′-end with biotin as a detector molecule is also prepared. The solutions are placed in a microplate, comprised of multiple wells, and spotting of the solutions on the inside of the lid of the substrate platform shown in FIG. 1, is carried out using conventional means such as for example, a BioChip Arrayer I from Packard BioChip Technologies.

Replicates of an array is spotted on the lid. Each array may be comprised of for example, about a row of 4 markers at the top and at the bottom to indicate the outer boundaries of the array, and in the middle rows, for example, about 10 middle rows, the capture probes of each SNP are printed in duplicates. Thus, a user may have a total of at least about 10 replicates of each capture probe. The arrays on the lid are spotted so that they will be positioned directly above the stretches of the meandering analysis chamber in the closed platform.

The spotted lid is irradiated for at least about 90 seconds, via conventional methods, such as for example, a Stratalinker 2400 from Stratagene, to allow the capture probes to form a covalent bond to the polymer lid, discussed in detail in the example which follows. After washing, the lid, which comprises the top section of the substrate platform, is placed on the base, or bottom section, of the closed platform substrate and attached by transmission laser welding, or any other means, to form airtight closed channels and chambers.

For each of the different alleles in the SNP's capture probes, a synthetic DNA oligomer is synthesized and labeled with biotin in the 5′-end (“Targets”). Conventional methods are used for synthesizing the oligomer such as, a DNA-synthesizer which can be purchased commercially. A solution of the targets in an appropriate buffer solution ( see, for example, Maniatis) is prepared at the desired concentration and is introduced into the inlet or sample port as shown e.g. in FIG. 1. The sample flows through the chambers as described above. The platform is left to hybridize over night at room temperature.

After hybridization the target solution is flushed out of the analysis chamber by applying, for example, at least about 200 μL of air through the sample port with a standard pipette. Subsequently, a solution of Cy5-labled streptavidin is added through the sample port completely filling the analysis chamber, and the platform is then left to incubate for 1 hour at room temperature.

Hybridization is observed due to, for example, the biotin label, allowing images to be produced using for example, a fluorescent microscope equipped with an XBO lamp, an emission/excitation filter set of 650 nm/670 nm and a 5×objective. Thus one can detect variations in single nucleotide polymorphisms.

The slides or substrate platforms of the invention may be used for any application which typically utilizes a standard microscope slide. For example, the slides may be used for evaluation of samples such as smears, sections, liquid samples, etc. The samples are preferably applied to the analysis area of the slide. The slides of the invention may be used in conjunction with any type of equipment, instrument or machine typically used to manipulate or evaluate a standard microscope slide.

The slides or substrate platforms of the invention may also be used for binding or immobilizing biomolecules. Biomolecules are preferably bound to the analysis area of the slide. The term ‘biomolecule’ as used herein is meant to indicate any type of nucleic acid, modified nucleic acid, protein, modified protein, peptide, modified peptide, small molecule, lectin, polysaccharide, hormone, drug, drug candidate, etc. Biomolecule binding may be covalent, non-covalent, direct, indirect, via a linker, targeted, random, etc. Biomolecules may be attached through a single attachment to the surface of the substrate platform or via multiple attachments for a single biomolecule. Any type of binding method known to the skilled in the art may be used.

Nucleic acids which may be immobilized onto the substrate include RNA, mRNA, DNA, LNA, PNA, cDNA, oligonucleotides, primers, nucleic acid binding partners, etc. The nucleic acids for immobilization may be modified by any method known in the art. For example, the nucleic acids may contain one or more modified nucleotides, etc. and/or one or more modified internucleotide linkages, such as, phosphorothioate, etc. Particularly preferred 3′ and/or 5′ modifications include amino modifiers, thiols, and photoreactive ketones particularly quinones, especially anthraquinones.

Particularly preferred modified nucleic acids are those containing one or more nucleoside analogues of the locked nucleoside analogue (LNA) type as described in WO 99/14226, which is incorporated herein by reference. Additionally, the nucleic acids may be modified at either the 340 and/or 5′ end by any type of modification known in the art. For example, either or both ends may be capped with a protecting group, attached to a flexible linking group, attached to a reactive group to aid in attachment to the substrate surface, etc.

As disclosed in WO 99/14226, LNA are a novel class of DNA analogues that form DNA- or RNA-heteroduplexes with exceptionally high thermal stability. LNA monomers include bicyclic compounds as shown immediately below:

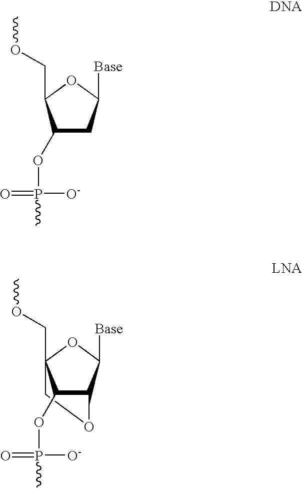

References herein to Locked Nucleoside Analogues, LNA or similar term refers to such compounds as disclosed in WO 99/14226.

LNA monomers and oligomers can share chemical properties of DNA and RNA; they are water soluble, can be separated by agarose gel electrophoresis, can be ethanol precipitated, etc.

Introduction of LNA monomers into either DNA, RNA or pure LNA oligonucleotides results in extremely high thermal stability of duplexes with complimentary DNA or RNA, while at the same time obeying the Watson-Crick base pairing rules. In general, the thermal stability of heteroduplexes is increased 3-8° C. per LNA monomer in the duplex. Oligonucleotides containing LNA can be designed to be substrates for polymerases (e.g. Taq polymerase), and PCR based on LNA primers is more discriminatory towards single base mutations in the template DNA compared to normal DNA-primers (i.e. allele specific PCR). Furthermore, very short LNA oligos (e.g. 8-mers) which have high Tₘ's when compared to similar DNA oligos, can be used as highly specific catching probes with outstanding discriminatory power towards single base mutations (i.e. SNP detection).

Oligonucleotides containing LNA are easily synthesized by standard phosphoramidite chemistry. The flexibility of the phosphoramidite synthesis approach further facilitates the easy production of LNA oligos carrying all types of standard linkers, fluorophores and reporter groups.

Particularly preferred LNA monomer for incorporation into an oligonucleotide for immobilization on the closed substrate analysis platform include those of the following formula Ia

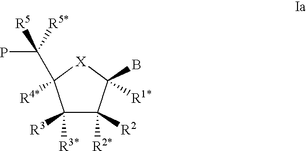

wherein X oxygen, sulfur, nitrogen, substituted nitrogen, carbon and substituted carbon, and preferably is oxygen; B is a nucleobase; R¹* , R², R³, R₅and R⁵* are hydrogen; P designates the radical position for an internucleoside linkage to a succeeding monomer, or a 5′-terminal group, R³* is an internucleoside linkage to a preceding monomer, or a 3′-terminal group; and R²* and R⁴* together designate —O—CH₂— where the oxygen is attached in the 2′-position, or a linkage of —CH₂)ₙ— where n is 2, 3 or 4, preferably 2, or a linkage of —S—CH₂— or —NH—CH₂—.

Units of formula Ia where R²* and R⁴* contain oxygen are sometimes referred to herein as “oxy-LNA”; units of formula Ia where R²* and R⁴* contain sulfur are sometimes referred to herein as “thio-LNA”; and units of formula Ia where R²* and R⁴* contain nitrogen are sometimes referred to herein as “amino-LNA”. For many applications, oxy-LNA units are preferred modified nucleic acid residues of oligonucleotides of the invention.

As used herein, including with respect to formula Ia, the term “nucleobase” covers the naturally occurring nucleobases adenine (A), guanine (G), cytosine (C), thymine (T) and uracil (U) as well as non-naturally occurring nucleobases such as xanthine, diaminopurine, 8-oxo-N⁶-methyladenine, 7-deazaxanthine, 7-deazaguanine, N⁴,N⁴-ethanocytosin, N⁶,N⁶-ethano-2,6-diaminopurine, 5-methylcytosine, 5-(C³-C⁶)-alkynyl-cytosine, 5-fluorouracil, 5-bromouracil, pseudoisocytosine, 2-hydroxy-5-methyl-4-triazolopyridin, isocytosine, isoguanine, inosine and the “non-naturally occurring” nucleobases described in Benner et al., U.S. Pat No. 5,432,272 and Susan M. Freier and Karl-Heinz Altmann, Nucleic Acids Research, 1997, vol. 25, pp 4429-4443. The term “nucleobase” thus includes not only the known purine and pyrimidine heterocycles, but also heterocyclic analogues and tautomers thereof. It should be clear to the person skilled in the art that various nucleobases which previously have been considered “non-naturally occurring” have subsequently been found in nature.

A “non-oxy-LNA” monomer is broadly defined as any nucleoside (i.e. a glycoside of a heterocyclic base) which does not contain an oxygen atom in a 2′-4′-sugar linkage. Examples of non-oxy-LNA monomers include 2′-deoxynucleotides (DNA) or nucleotides (RNA) or any analogues of these monomers which are not oxy-LNA, such as for example the thio-LNA and amino-LNA described above with respect to formula 1a and in Singh et al. J. Org. Chem. 1998, 6, 6078-9, and the derivatives described in Susan M. Freier and Karl-Heinz Altmann, Nucleic Acids Research, 1997, vol 25, pp 4429-4443.

A wide variety of modified nucleic acids may be employed, including those that have 2′-modification of hydroxyl, 2′-O-methyl, 2′-fluoro, 2′-trifluoromethyl, 2′-O-(2-methoxyethyl), 2-O-aminopropyl, 2′-O-dimethylamino-oxyethyl, 2′-O-fluoroethyl or 2′-O-propenyl. The nucleic acid may further include a 3′ modification, preferably where the 2′- and 3′-position of the ribose group is linked. The nucleic acid also may contain a modification at the 4′-position, preferably where the 2′- and 4′-positions of the ribose group are linked such as by a 2′-4′ link of —CH₂—S—, —CH₂—NH—, or —CH₂—NMe— bridge.

The nucleotide also may have a variety of configurations such as α-D-ribo, β-D-xylo, or α-L-xylo configuration.

The internucleoside linkages of the residues of oligos of the invention may be natural phosphorodiester linkages, or other linkages such as —O—P(O)₂—O—, —O—P(O,S)—O—, —O—P(S)₂—O—,—NR^{H}—P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl.

A further preferred group of modified nucleic acids for incorporation into oligomers of the invention include those of the following formula:

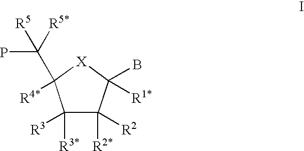

wherein

X is —O—; B is selected from nucleobases; R¹* is hydrogen; P designates the radical position for an internucleoside linkage to a succeeding monomer, or a 5′-terminal group, such internucleoside linkage or 5′-terminal group optionally including the substituent R⁵, R⁵ being hydrogen or included in an internucleoside linkage, R³* is a group P* which designates an internucleoside linkage to a preceding monomer, or a 3′-terminal group; one or two pairs of non-geminal substituents selected from the present substituents of R², R²* , R³, R⁴* , may designate a biradical consisting of 1-4 groups/atoms selected from —C(R^{a}R^{b})—, —C(R^{a})═C(R^{a})—, —C(R^{a})═N—, —O—, —S—, —SO₂—, —N(R^{a})—, and >C=Z, wherein Z is selected from —O—, —S—, and —N(R^{a})—, and R^{a} and R^{b} each is independently selected from hydrogen, optionally substituted C₁₋₆-alkyl, optionally substituted C²⁻⁶-alkoxycarbonyl, C₁₋₆-alkenyl, hydroxy, C₁₋₆-alkoxy, C₂₋₆-alkenyloxy, carboxy, C₁₋₆-alkoxycarbonyl, C₁₋₆-alkylcarbonyl, formyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino -C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, photochemically active groups, thermochemically active groups, chelating groups, reporter groups, and ligands,

said possible pair of non-geminal substituents thereby forming a monocyclic entity together with (i) the atoms to which said non-geminal substituents are bound and (ii) any intervening atoms; and

each of the substituents R², R²* , R³, R⁴* which are present and not involved in the possible biradical is independently selected from hydrogen, optionally substituted C₁₋₆-alkyl, optionally substituted C₂₋₆-alkenyl, hydroxy, C₁₋₆-alkoxy, C₂₋₆-alkenyloxy, carboxy, C₁₋₆-alkoxycarbonyl, C₁₋₆-alkylcarbonyl, formyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, photochemically active groups, thermochemically active groups, chelating groups, reporter groups, and ligands; and basic salts and acid addition salts thereof.

Particularly preferred LNA monomers for use in the closed substrate analysis platform are 2′-deoxyribonucleotides, ribonucleotides, and analogues thereof that are modified at the 2′-position in the ribose, such as 2′-O-methyl, 2′-fluoro, 2′-trifluoromethyl, 2′-O-(2-methoxyethyl), 2′-O-aminopropyl, 2′-O-dimethylamino-oxyethyl, 2′-O-fluoroethyl or 2′-O-propenyl, and analogues wherein the modification involves both the 2′ and 3′0 position, preferably such analogues wherein the modifications links the 2′- and 3′-position in the ribose, such as those described in Nielsen et al., J. Chem. Soc., Perkin Trans. 1, 1997, 3423-33, and in WO 99/14226, and analogues wherein the modification involves both the 2′- and 4′-position, preferably such analogues wherein the modifications links the 2′- and 4′-position in the ribose, such as analogues having a —CH₂—S— or a —CH₂—NH— or a —CH₂—NMe— bridge (see Singh et al. J. Org. Chem. 1998, 6, 6078-9). Although LNA monomers having the β-D-ribo configuration are often the most applicable, other configurations also are suitable for purposes of the invention. Of particular use are α-L-ribo, the β-D-xylo and the α-L-xylo configurations (see Beier et al., Science, 1999, 283, 699 and Eschenmoser, Science, 1999, 284, 2118), in particular those having a 2′-4′—CH₂—S—, —CH₂—NH—, —CH₂—O— or —CH₂—NMe— bridge.

In the present context, the term “oligonucleotide” which is the same as “oligomer” which is the same as “oligo” means a successive chain of nucleoside monomers (i.e. glycosides of heterocyclic bases) connected via internucleoside linkages. The linkage between two successive monomers in the oligo consist of 2 to 4, preferably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHRH)—, where R^{H} is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH═(including R⁵ when used as a linkage to a succeeding monomer), —CH₂—CH₂—O—, —NR^{H}—CH₂—CH₂—, —CH₂—CH₂—NR^{H}—, —CH₂—NR^{H}—CH₂—, —O—CH₂—CH₂—NR^{H}—, —NR^{H}—CO—O—, —NR^{H}—CO—NR^{H}—, —NR^{H}—CS—NR^{H}—, —NR^{H}—C(═NR^{H})—NR^{H}—, —NR^{H}—CO—CH₂—NR^{H}—, —O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—O—, —CH₂—O—N═(including R⁵ when used as a linkage to a succeeding monomer), —CH₂—O—NR^{H}—, —CO—NR^{H}—CH₂—, —CH₂—, NR^{H}—O—, —CH₂—NR^{H}—CO—, —O—NR^{H}—CH₂—, —O—NR^{H}—, —O—CH₂—S—, —S—CH₂—O—, —CH₂—CH₂—S—, —O—CH₂—CH₂—S—, —S—CH₂—CH═(including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—C₂—, —O—SO—O—,—O—S(O)₂—O—, —O—S(O)₂—CH₂—, —O—S(O)₂—NR^{H}—, —NR^{H}—S(O)₂—CH₂—, —O—S(O)₂—CH₂—, —O—P(O)₂—O—, —O—P(O,S)—O—, —O—P(S)₂—O—,—S—P(O)₂—O—, —S—P(O,S)—O—,—S—, P(S)₂—O—, —O—P(O)₂—S—, —O—P(O,S)—S—, —O—P(S)₂—S—, —S—P(O)₂—S—, —S—P(O,S)—S—, —S—P(S)₂—S—, ——O—PO(R″)—O—, —O—PO(OCH₃)—O—, —O—PO(OCH₂CH₃)—O—, —O—PO(OCH₂CH₂S—R)—O—, —O—PO(BH₃)—O—, —O—PO(NHR^{N})—O—, —O—P(O)₂—NR^{H}—, —NR^{H}P(O)₂—O—, —O—P(O,NR^{H})—O—, —CH₂—P(O)₂—O—, —O—P(O)₂—CH₂—, and —O—Si(R″)₂—O—; among which —CH₂—CO—NR^{H}—, —CH₂—NR^{H}—O—, —S—CH₂—O—, —O—P(O)₂—O—, —O—P(O,S)—O—, —O—P(S)₂—O—, —NR^{H}—P(O)₂—O—, —O—P(O,NR^{H})—O—, —O—PO(R″)—O—, —O—PO(CH₃)—O—, and —O—PO(NHR^{N})—O—, where R^{H} is selected form hydrogen and C₁₋₄-alkyl, and R″ is selected from C₁₋₆-alkyl and phenyl, are especially preferred. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443. The left-hand side of the internucleoside linkage is bound to the 5-membered ring as substituent P* at the 3′ position, whereas the right-hand side is bound to the 5′-position of a preceding monomer.

The term “succeeding monomer” relates to the neighboring monomer in the 5′-terminal direction and the “preceding monomer” relates to the neighboring monomer in the 3′-terminal direction.

Monomers are referred to as being “complementary” if they contain nucleobases that can form hydrogen bonds according to Watson-Crick base-pairing rules (e.g. G with C, A with T or A with U) or other hydrogen bonding motifs such as for example diaminopurine with T, inosine with C, pseudoisocytosine with G, etc.

An “LNA modified oligonucleotide” is used herein to describe oligonucleotides comprising at least one LNA monomeric residue of the general scheme A, described infra, having the below described illustrative examples of modifications:

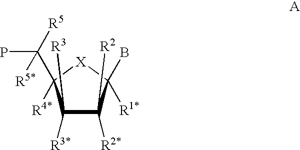

wherein

X is selected from —O—, —S—, —N(R^{N})—, —C(R⁶R⁶*)—, —O—C(R⁷R⁷*)—, —C(R⁶R⁶*)—O—, —S—C(R⁷R⁷*)—, —C(R⁶R⁶*)—S—, —N(R^{N}*)—C(R⁷R⁷*)—, —C(R⁶R⁶*)—N(R^{N}*)—, and —C(R⁶R⁶*) —C(R⁷R⁷*)—;

B is selected from hydrogen, hydroxy, optionally substituted C₁₋₄-alkoxy, optionally substituted C¹⁻⁴-alkyl, optionally substituted C₁₋₄-acyloxy, nucleobases, DNA intercalators, photochemically active groups, thermochemically active groups, chelating groups, reporter groups, and ligands;

P designates the radical position for an internucleoside linkage to a succeeding monomer, or a 5′-terminal group, such internucleoside linkage or 5′-terminal group optionally including the substituent R⁵;

one of the substituents R², R²*, R³, and R³* is a group P* which designates an internucleoside linkage to a preceding monomer, or a 2′/3′-terminal group;

the substituents of R¹*, R⁴*, R⁵, R⁵*, R⁶, R⁶*, R⁷, R⁷*, R^{N}, and the ones of R², R²*, R³, and R³* not designating P* each designates a biradical comprising about 1-8 groups/atoms selected from —C(R^{a}R^{b})—, —C(R^{a})═C(R^{a})—, —C(R^{a})═N—, —C(R^{a})—O—, —O—, —Si(R^{a})₂—, —C(R^{a})—S, —S—, —SO₂—, —C(R^{a})—N(R^{b})—, —N(R^{a})—, and >C═Q,

wherein Q is selected from —O—, —S—, and —N(R^{a})—, and R^{a} and R^{b} each is independently selected from hydrogen, optionally substituted C₁₋₁₂-alkyl, optionally substituted C₂₋₁₂-alkenyl, optionally substituted C₂₋₁₂-alkynyl, hydroxy, C₂₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents R^{a} and R^{b} together may designate optionally substituted methylene (═CH₂), and wherein two non-geminal or geminal substituents selected from R^{a}, R^{b}, and any of the substituents R¹*, R², R²* , R³, R³*, R⁴*, R⁵, R⁵*, R⁶ and R⁶,* R⁷, and R⁷* which are present and not involved in P, P* or the biradical(s) together may form an associated biradical selected from biradicals of the same kind as defined before;

said pair(s) of non-geminal substituents thereby forming a mono- or bicyclic entity together with (i) the atoms to which said non-geminal substituents are bound and (ii) any intervening atoms; and

each of the substituents R¹*, R², R²*, R³, R⁴*, R⁵, R⁵*, R⁶ and R⁶*, R⁷, and R⁷* which are present and not involved in P, P* or the biradical(s), is independently selected from hydrogen, optionally substituted C₁₋₁₂-alkyl, optionally substituted C₂₋₁₂-alkenyl, optionally substituted C₂₋₁₂-alkynyl, hydroxy, C₁₋₁₂-alkoxy, C₂₋₁₂-alkenyloxy, carboxy, C₁₋₁₂-alkoxycarbonyl, C₁₋₁₂-alkylcarbonyl, formyl, aryl, aryloxy-carbonyl, aryloxy, arylcarbonyl, heteroaryl, heteroaryloxy-carbonyl, heteroaryloxy, heteroarylcarbonyl, amino, mono- and di(C₁₋₆-alkyl)amino, carbamoyl, mono- and di(C₁₋₆-alkyl)-amino-carbonyl, amino-C₁₋₆-alkyl-aminocarbonyl, mono- and di(C₁₋₆-alkyl)amino-C₁₋₆-alkyl-aminocarbonyl, C₁₋₆-alkyl-carbonylamino, carbamido, C₁₋₆-alkanoyloxy, sulphono, C₁₋₆-alkylsulphonyloxy, nitro, azido, sulphanyl, C₁₋₆-alkylthio, halogen, DNA intercalators, photochemically active groups, thermochemically active groups, chelating groups, reporter groups, and ligands, where aryl and heteroaryl may be optionally substituted, and where two geminal substituents together may designate oxo, thioxo, imino, or optionally substituted methylene, or together may form a spiro biradical consisting of a 1-5 carbon atom(s) alkylene chain which is optionally interrupted and/or terminated by one or more heteroatoms/groups selected from —O—, —S—, and —(NR^{N})— where R^{N} is selected from hydrogen and C₁₋₄-alkyl, and where two adjacent (non-geminal) substituents may designate an additional bond resulting in a double bond; and R^{N}*, when present and not involved in a biradical, is selected from hydrogen and C₁₋₄-alkyl; and basic salts and acid addition salts thereof,

In another preferred embodiment, LNA modified oligonucleotides used in closed analysis substrate platform comprises oligonucleotides containing at least one LNA monomeric residue of the general scheme A above:

wherein X, B, P are defined as above;

one of the substituents R², R²*, R³, and R³* is a group P* which designates an internucleoside linkage to a preceding monomer, or a 2′/3′-terminal group;

substituent together designates a biradical structure selected from -(CR*R*)ₜ-M-(CR*R*)ₛ—, —(CR*R*)ᵣ-M-(CR*R*)ₛ-M-, -M-(CR*R*)ᵣ₊ₛ-M- -M-(CR*R*)ᵣ-M-(CR*R*)ₛ—, —(CR*R*)ᵣ₊ₛ—, -M-, -M-M-, wherein each M is independently selected from —O—, —S—, —Si(R*)₂—, —N(R*)—, >C═O, —C(═O)—N(R*)-, and —N(R*)—C(═O)—. Each R* and R^{1(1*)}—R^{7(7*)}, which are not involved in the biradical, are independently selected from hydrogen, halogen, azido, cyano, nitro, hydroxy, mercapto, amino, mono- or di(C₁₋₆-alkyl)amino, optionally substituted C₁₋₆-alkoxy, optionally substituted C₁₋₆-alkyl, DNA intercalators, photochemically active groups, thermochemically active groups, chelating groups, reporter groups, and ligands, and/or two adjacent (non-geminal) R* may together designate a double bond, and each of r and s is 0-4 with the proviso that the sum r+s is 1-5.

In a most preferred embodiment LNA-nucleoside conjugates used in the closed substrate analysis platform comprise nucleosides containing at least one LNA monomeric residue of the general formula shown scheme B:

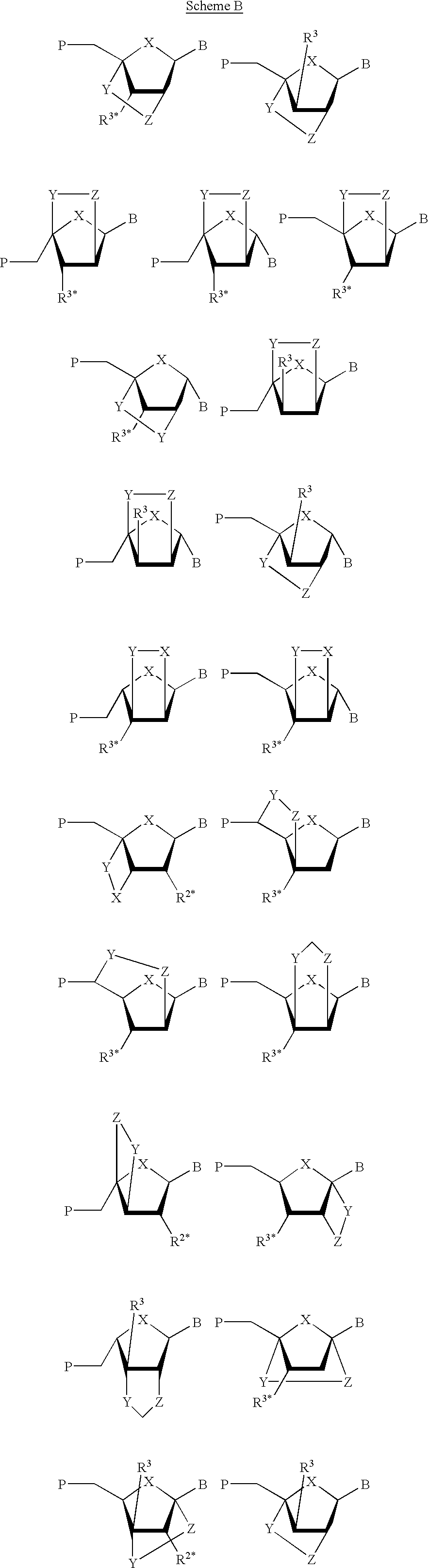

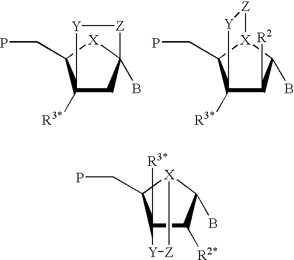

Wherein the groups, X and B are defined as above.

P designates the radical position for an internucleoside linkage to a succeeding monomer, nucleoside such as an L-nucleoside, or a 5′-terminal group, such internucleoside linkage or 5′-terminal group optionally including the substituent R⁵;

one of the substituents R², R²*, R³, and R³* is a group P* which designates an internucleoside linkage to a preceding monomer, or a 2′/3′-terminal group;

Preferred nucleosides are L-nucleosides such as for example, derived dinucleoside monophosphates. The nucleoside can be comprised of either a beta-D, a beta-L or an alpha.-L nucleoside. Preferred nucleosides may be linked as dimers wherein at least one of the nucleosides is a beta-L or alpha-L. B may also designate the pyrimidine bases cytosine, thymine, uracil, or 5-fluorouridine (5-FUdR) other 5-halo compounds, or the purine bases, adenosine, guanosine or inosine.

The chimeric oligos for use in the closed substrate analysis platform are highly suitable for a variety of diagnostic purposes such as for the isolation, purification, amplification, detection, identification, quantification, or capture of nucleic acids such as DNA, mRNA or non-protein coding cellular RNAs, such as tRNA, rRNA, snRNA and scRNA, or synthetic nucleic acids, in vivo or in vitro. The use of any of the oligomers described herein, for immobilization onto the closed substrate analysis platform allows for a variety of important uses as seen below.

The oligomer can comprise a photochemically active group, a thermochemically active group, a chelating group, a reporter group, or a ligand that facilitates the direct of indirect detection of the oligomer or the immobilization of the oligomer onto a solid support. Such group are typically attached to the oligo when it is intended as a probe for in situ hybridization, in Southern hybridization, Dot blot hybridization, reverse Dot blot hybridization, or in Northern hybridization.

When the photochemically active group, the thermochemically active group, the chelating group, the reporter group, or the ligand includes a spacer (K), the spacer may suitably comprise a chemically cleavable group.

In the present context, the term “photochemically active groups” covers compounds which are able to undergo chemical reactions upon irradiation with light. Illustrative examples of functional groups hereof are quinones, especially 6-methyl-1,4-naphtoquinone, anthraquinone, naphtoquinone, and 1,4-dimethyl-anthraquinone, diazirines, aromatic azides, benzophenones, psoralens, diazo compounds, and diazirino compounds.

In the present context “thermochemically reactive group” is defined as a functional group which is able to undergo thermochemically-induced covalent bond formation with other groups. Illustrative examples of functional parts thermochemically reactive groups are carboxylic acids, carboxylic acid esters such as activated esters, carboxylic acid halides such as acid fluorides, acid chlorides, acid bromide, and acid iodides, carboxylic acid azides, carboxylic acid hydrazides, sulfonic acids, sulfonic acid esters, sulfonic acid halides, semicarbazides, thiosemicarbazides, aldehydes, ketones, primary alcohols, secondary alcohols, tertiary alcohols, phenols, alkyl halides, thiols, disulphides, primary amines, secondary amines, tertiary amines, hydrazines, epoxides, maleimides, and boronic acid derivatives.

In the present context, the term “chelating group” means a molecule that contains more than one binding site and frequently binds to another molecule, atom or ion through more than one binding site at the same time. Examples of functional parts of chelating groups are iminodiacetic acid, nitrilotriacetic acid, ethylenediamine tetraacetic acid (EDTA), aminophosphonic acid, etc.

In the present context, the term “reporter group” means a group which is detectable either by itself or as a part of an detection series. Examples of functional parts of reporter groups are biotin, digoxigenin, fluorescent groups (groups which are able to absorb electromagnetic radiation, e.g. light or X-rays, of a certain wavelength, and which subsequently reemits the energy absorbed as radiation of longer wavelength; illustrative examples are dansyl (5-dimethylamino)-1-naphthalenesulfonyl), DOXYL (N-oxyl-4,4-dimethyloxazolidine), PROXYL (N-oxyl-2,2,5,5-tetramethylpyrrolidine), TEMPO (N-oxyl-2,2,6,6-tetramethylpiperidine), dinitrophenyl, acridines, coumarins, Cy3 and Cy5 (trademarks for Biological Detection Systems, Inc.), erythrosine, coumaric acid, umbelliferone, Texas red, rhodamine, tetramethyl rhodamine, Rox, 7-nitrobenzo-2-oxa-1-diazole (NBD), pyrene, fluorescein, Europium, Ruthenium, Samarium, and other rare earth metals), radioisotopic labels, chemiluminescence labels (labels that are detectable via the emission of light during a chemical reaction), spin labels (a free radical (e.g. substituted organic nitroxides) or other paramagnetic probes (e.g. Cu²⁺, Mg²⁺) bound to a biological molecule being detectable by the use of electron spin resonance spectroscopy), enzymes (such as peroxidases, alkaline phosphatases, p-galactosidases, and glycose oxidases), antigens, antibodies, haptens (groups which are able to combine with an antibody, but which cannot initiate an immune response by itself, such as peptides and steroid hormones), carrier systems for cell membrane penetration such as: fatty acid residues, steroid moieties (cholesteryl), vitamin A, vitamin D, vitamin E, folic acid peptides for specific receptors, groups for mediating endocytose, epidermal growth factor (EGF), bradykinin, and platelet derived growth factor (PDGF). Especially interesting examples are biotin, fluorescein, Texas Red, rhodamine, dinitrophenyl, digoxigenin, Ruthenium, Europium, Cy5, Cy3, etc.

In the present context “ligand” refers to the binding of a first molecule to another molecule which has an affinity for the first molecule, such as for example a TNF molecule (ligand) binding to the TNF receptor. Ligands can comprise functional groups such as: aromatic groups (such as benzene, pyridine, naphthalene, anthracene, and phenanthrene), heteroaromatic groups (such as thiophene, furan, tetrahydrofuran, pyridine, dioxane, and pyrimidine), carboxylic acids, carboxylic acid esters, carboxylic acid halides, carboxylic acid azides, carboxylic acid hydrazides, sulfonic acids, sulfonic acid esters, sulfonic acid halides, semicarbazides, thiosemicarbazides, aldehydes, ketones, primary alcohols, secondary alcohols, tertiary alcohols, phenols, alkyl halides, thiols, disulphides, primary amines, secondary amines, tertiary amines, hydrazines, epoxides, maleimides, C₁₋₂₀ alkyl groups optionally interrupted or terminated with one or more heteroatoms such as oxygen atoms, nitrogen atoms, and/or sulphur atoms, optionally containing aromatic or mono/polyunsaturated hydrocarbons, polyoxyethylene such as polyethylene glycol, oligo/polyamides such as poly-a-alanine, polyglycine, polylysine, peptides, oligo/polysaccharides, oligo/polyphosphates, toxins, antibiotics, cell poisons, and steroids, and also “affinity ligands”, ie. functional groups or biomolecules that have a specific affinity for sites on particular proteins, antibodies, poly- and oligosaccharides, and other biomolecules.

It should be understood that the above-mentioned specific examples under DNA intercalators, photochemically active groups, thermochemically active groups, chelating groups, reporter groups, and ligands correspond to the “active/functional” part of the groups in question. For the person skilled in the art it is furthermore clear that DNA intercalators, photochemically active groups, thermochemically active groups, chelating groups, reporter groups, and ligands are typically represented in the form M-K- where M is the “active/functional” part of the group in question and where K is a spacer through which the “active/functional” part is attached to the 5- or 6-membered ring. Thus, it should be understood that the group B, in the case where B is selected from DNA intercalators, photochemically active groups, thermochemically active groups, chelating groups, reporter groups, and ligands, has the form M-K-, where M is the “active/functional” part of the DNA intercalator, photochemically active group, thermochemically active group, chelating group, reporter group, and ligand, respectively, and where K is an optional spacer comprising 1-50 atoms, preferably 1-30 atoms, in particular 1-15 atoms, between the 5- or 6-membered ring and the “active/functional” part.

In the present context, the term “spacer” means a thermochemically and photochemically non-active distance-making group and is used to join two or more different moieties of the types defined above. Spacers are selected on the basis of a variety of characteristics including their hydrophobicity , hydrophilicity, molecular flexibility and length (e.g. see Hermanson et. al., “Immobilized Affinity Ligand Techniques”, Academic Press, San Diego, Calif. (1992), p. 137-ff). Generally, the length of the spacers are less than or about 400 Å, in some applications preferably less than 100 Å. The spacer, thus, comprises a chain of carbon atoms optionally interrupted or terminated with one or more heteroatoms, such as oxygen atoms, nitrogen atoms, and/or sulphur atoms. Thus, the spacer K may comprise one or more amide, ester, amino, ether, and/or thioether functionalities, and optionally aromatic or mono/polyunsaturated hydrocarbons, polyoxyethylene such as polyethylene glycol, oligo/polyamides such as poly-α-alanine, polyglycine, polylysine, and peptides in general, oligosaccharides, oligo/polyphosphates. Moreover the spacer may consist of combined units thereof. The length of the spacer may vary, taking into consideration the desired or necessary positioning and spatial orientation of the “active/functional” part of the group in question in relation to the 5- or 6-membered ring. In particularly interesting embodiments, the spacer includes a chemically cleavable group. Examples of such chemically cleavable groups include disulphide groups cleavable under reductive conditions, peptide fragments cleavable by peptidases, etc.

As discussed above, these oligonucleotides may be used in the closed substrate analysis platform for the construction of high specificity oligo arrays e.g. wherein a multitude of different oligos are affixed to a solid surface in a predetermined pattern (Nature Genetics, suppl. vol. 21, Jan 1999, 1-60 and WO 96/31557). The usefulness of such an array, which can be used to simultaneously analyze a large number of target nucleic acids, depends to a large extend on the specificity of the individual oligos bound to the surface. The target nucleic acids may carry a detectable label or be detected by incubation with suitable detection probes which may also be an oligonucleotide of the invention.

An illustrative example for use of a closed substrate analysis platform is for identification of a nucleic acid sequence capable of binding to a biomolecule of interest. This is achieved by immobilizing a library of nucleic acids onto the substrate surface so that each unique nucleic acid is located at a defined position to form an array. The array is then exposed to the biomolecule under conditions which favor binding of the biomolecule to the nucleic acids. Non-specifically binding biomolecules are washed away using mild to stringent buffer conditions depending on the level of specificity of binding desired. The nucleic acid array is then analyzed to determine which nucleic acid sequences bound to the biomolecule. Preferably the biomolecules would carry a fluorescent tag for use in detection of the location of the bound nucleic acids.

The closed substrate platforms, with an immobilized array of nucleic acid sequences may be used for determining the sequence of an unknown nucleic acid; single nucleotide polymorphism (SNP) analysis; analysis of gene expression patterns from a particular species, tissue, cell type, etc.; gene identification; etc.

Nucleic acids for immobilization onto the substrate may be either single stranded or double stranded and preferably contain from about 2 to about 1000 nucleotides, more preferably from about to 2 to about 100 nucleotides and most preferably from about 2 to about 30 nucleotides.

Polypeptides may also be immobilized onto the surface of the substrate platform. Particularly preferred polypeptides for immobilization are receptors, ligands, antibodies, antigens, enzymes, nucleic acid binding proteins, etc. Polypeptides may be modified in any way known to those skilled in the art. For example, polypeptides may contain one or more phosphorylations, glycosylations, etc. Additionally, polypeptides may be attached to a flexible linker and/or reactive to group to facilitate binding to the surface of the substrate.

Polypeptides for immobilization onto the substrate may be monomeric, dimeric or multimeric and preferably contain from about 2 to about 1000 amino acids, more preferably from about 2 to about 100 amino acids and most preferably from about 2 to about 20 amino acids.

Polypeptides and nucleic acids for immobilization onto the substrate may be prepared separately and then applied onto the substrate surface. Methods for preparation of nucleic acids/oligos are known in the art, for example phosphoramidite chemistry.

Polypeptides and nucleic acids may be applied to the surface of the substrate by any method well known in the art. For example, polypeptides or nucleic acids may be manually pipetted onto the surface or applied using a robotics system. Preferably, polypeptides or nucleic acids are applied to the substrate using a micro spotting technique such as may be achieved with inkjet type technology.

The analysis substrates of the invention also may be employed for relatively high density analysis, e.g. loaded for analysis with at least about 100 unique polypeptide sequences or nucleotides sequences per cm²of analysis area; or at least about 200, 300, 400, 500, 600, 700, 800 or 900 unique polypeptide sequences or nucleotides sequences per cm²of analysis area.

Biomolecules may be attached to the surface of the substrate using any method known in the art. Preferably biomolecules are attached to the surface using a photochemical linker which becomes active upon exposure to light of a defined wavelength. Most preferably biomolecules are attached to the surface using a quinone photolinker. Methods for photochemical immobilization of biomolecules using quinones are described in WO 96/31557, which is incorporated herein by reference.

Biomolecules may be attached directly to the analysis substrate surface or may be attached to the substrate through a flexible linker group. The linker group may be attached to the surface of the substrate before immobilization of the biomolecule or the linker group may be attached to the biomolecule before immobilization onto the substrate. For example, a nucleic acid may be modified with a linker group at either the 3′ or 5′ end prior to immobilization onto the substrate. Alternatively, an unmodified nucleic acid may be attached to the substrate which has been coated with linker groups. Similarly, a polypeptide may be modified with a group at either the amino terminus or carboxy terminus prior to immobilization onto the substrate. Alternatively, an unmodified polypeptide may be immobilized onto the substrate which has been coated with linker groups. The linker groups may be attached at any location within a nucleic acid or polypeptide chain but are preferably attached at either end of the polypeptide or amino acid chain. Linker groups for immobilization of biomolecules are well known in the art. Any linker group known in the art may be used for attachment of biomolecules.

Alternatively, polypeptides and nucleic acids may be synthesized in situ on the surface of the substrate. Methods for in situ synthesis of polypeptides and nucleic acids are well known in the art and include photolithographic techniques, protection/deprotection techniques, etc.

The analysis area of the substrate platforms of the invention may be coated with a single biomolecule, with a random mixture of biomolecules or with a mixture of biomolecules wherein each unique biomolecule is located at a defined position so as to form an array. In a preferred embodiment the analysis area is coated with a library of polypeptides or nucleic acids wherein each unique nucleic acid or amino acid sequence is located at a defined location within the analysis area.

The invention also provides methods for using the substrate platforms of the invention for carrying out a variety of bioassays. Any type of assay wherein one component is immobilized may be carried out using the substrate platforms of the invention. Bioassays utilizing an immobilized component are well known in the art. Examples of assays utilizing an immobilized component include for example, immunoassays, analysis of protein-protein interactions, analysis of protein-nucleic acid interactions, analysis of nucleic acid-nucleic acid interactions, receptor binding assays, enzyme assays, phosphorylation assays, diagnostic assays for determination of disease state, genetic profiling for drug compatibility analysis, SNP detection, etc.

Identification of a nucleic acid sequence capable of binding to a biomolecule of interest could be achieved by immobilizing a library of nucleic acids onto the substrate surface so that each unique nucleic acid was located at a defined position to form an array. The array would then be exposed to the biomolecule under conditions which favored binding of the biomolecule to the nucleic acids. Non-specifically binding biomolecules could be washed away using mild to stringent buffer conditions depending on the level of specificity of binding desired. The nucleic acid array would then be analyzed to determine which nucleic acid sequences bound to the biomolecule. Preferably the biomolecules would carry a fluorescent tag for use in detection of the location of the bound nucleic acids.

Assays using an immobilized array of nucleic acid sequences may be used for determining the sequence of an unknown nucleic acid; single nucleotide polymorphism (SNP) analysis; analysis of gene expression patterns from a particular species, tissue, cell type, etc.; gene identification; gene deletion analysis, etc.

Assays using immobilized polypeptides are also provided by the methods of the invention. For example, an immobilized array of peptides could be exposed to an antibody or receptor to determine which peptides are recognized by the antibody or receptor. Preferably the antibody or receptor carriers a fluorescent tag for identification of the location of the bound peptides. Alternatively, an immobilized array of antibodies or receptors could be exposed to a polypeptide to determine which antibodies recognize the polypeptide.

The slides of the invention may also be used for assays not involving immobilized biomolecules. For example, the slides may be used for cell sorting, including living cells (inclusive of viruses), which sorted cells then may be subjected to analysis.

Analysis substrates of the invention also may be modified as appropriate for particular assays. For instance, in closed analysis systems of the invention, one or more surfaces of the internal analysis surface can be pre-treated to facilitate attachment and/or growth of cells for analysis.

All documents mentioned herein are incorporated herein by reference in their entirety.

The invention has been described in detail with reference to preferred embodiments thereof. However, it will be appreciated that those skilled in the art, upon consideration of this disclosure, may make modifications and improvements within the spirit and scope of the invention.

### EXAMPLE

### Hybridization Performed in Meander Design

To demonstrate the functionality of the closed platform substrate an assay was performed inside the analysis chamber, demonstrating the genotyping of 10 single nucleotide polymorphisms (SNP's) simultaneously. The discrimination between the alleles was based exclusively on hybridization efficiencies of 20 LNA-containing capture probes—one for each of the two alleles in 10 SNP's.

All the capture probes were of the same overall design, comprising in the 3 ′-end a 12-mer mixed sequence of DNA and LNA having a perfect match to one of the two alleles in a specific SNP. In the 5′-end of this 12-mer sequence a “t-15” linker consisting of 15 consecutive thymine DNA bases, is connecting the capturing part of the probe to a photo reactive anthraquinone according to patent no. WO 96/31557. The SNP and the two corresponding capture probes are listed in Table 1.

TABLE 1
The capture probes having sequences complementary to a target in
the hybridized mixture, and thus expected to show a high hybridization
signal, are marked with bold. (Small letters designating DNA-nucleotides
and capital letters designating LNA-nucleotides. All LNA C are methyl-C)

| SNP | CAPTURE PROBE 1 | CAPTURE PROBE 2 | HYBRIDIZED SYNTHETIC TARGET |
|---|---|---|---|
| Apo112 | GCcgCACacgtc | gCcgCGCacgTc | atggaggacgtgtgcggccgcctggtgcag |
| ApoE158 | caggCACttcTG | caggCGCttcTG | atgccgatgacctgcagaagtgcctggcag |
| ApoB71 | ttcagGATgcAC | ttcagGGTgcAC | accagccagtgcatcctgaaagaggtgtat |
| ApoB3500 | gaagaCTGtgTG | GAagaCCGtgtg | tccaagagcacacggtcttcagtgaagctg |
| ApoB3500-1 | GAagaCCGtgtg | aAgacCATgtGc | tccaagagcacacggtcttcagtgaagctg |
| HNF1-131 | acctcCCGcTGt | cCtccCACtgTg | gtcctacctgcagcagcacaacatcccacagcgggaggtggtcgatacca |
| HNF1-131-1 | aCctcCTGctGt | acctcCCGcTGt | gtcctacctgcagcagcacaacatcccacagcgggaggtggtcgatacca |
| HNF4-154 | cctgtCAGgaCA | cctgtCGGgaCA | ctgtcccgacaggtaccggggtgatcctgc |
| HNF4-276 | cataCTCAttGt | cataCTGAttGt | ggagctgcagatcgatgacaatgagtatgc |
| ApoB4154 | ggaatTTTtGAg | ggaatrCTtgAG | tacgagttactcaaaaattccatatgaaag |

10 μM solutions of the 20 capture probes, in 100 mM phosphate buffer, pH=7.2 were prepared. For alignment purposes a 1:M solution of a t-15 oligonucleotide modified in the 5′-end with anthraquinone and with biotin in the 3′-end was also prepared, in 100 mM phosphate buffer, pH=7.2. All solutions were placed in a 384-well microplate and spotted on the inside of the lid of the structure shown in FIG. 1, using a BioChip Arrayer I from Packard BioChip Technologies.

Five replicates of a 4×12 array were spotted onto the lid. Each array contained a row of 4 markers at the top and at the bottom to indicate the outer boundaries of the array, and in the 10 middle rows the two capture probes of each SNP were printed in duplicates—in total 10 replicates of each capture probe. The 5 arrays on the lid were spotted so that they would be positioned directly above the 5 stretches of the meandering analysis chamber in the closed platform.

The spotted lid was irradiated for 90 seconds in a Stratalinker 2400 from Stratagene. During the irradiation the photo reactive anthraquinone in the 5′-end of the capture probes formed a covalent bond to the polymer lid. After irradiation the lid was washed in highly purified water for 2 hours to remove salts from the spotting buffer and excess of capture probes not covalently coupled to the lid. After washing the lid was dried for 30 min. at 37° C.

The lid was then placed on the base of the closed platform substrate containing the microfluidics structure and attached by transmission laser welding to form closed channels and chambers.

For each of the two different alleles in the 10 SNP's a synthetic 30- or 50-mer DNA oligonucleotide labeled with biotin at the 5′-end was synthesized (“Targets”). A mixture of the 10 oligonucleotide targets (one for each SNP) serves as a model system for genotyping of a sample. A solution of the 10 selected targets listed in Table 1 was prepared in a buffer containing final concentrations of 600 mM NaCl, 60 mM Sodium Citrate and 0.1% (v/v) Tween20, pH=7.0 (4×SSCT). Each target oligonucleotide was present at a final concentration of 0.01 μM. 70 μL of this target solution was added to the sample port of the closed substrate platform, filling the complete analysis chamber. The platform was left to hybridize over night at room temperature. The combination of high salt concentration and low temperature corresponds to low stringency conditions.

After hybridization the target solution was flushed out of the analysis chamber by applying 200 μL of air through the sample port with a standard pipette. Subsequently 70 μL of a solution of Cy5-labled streptavidin 1 μg/mL in 5×SSCT was added through the sample port completely filling the analysis chamber, and the platform was then left to incubate for 1 hour at room temperature. Finally, the analysis chamber was still filled with the 0.15×SSCT washing buffer at room temperature.

Table 2 shows the hybridization of one of the arrays in the analysis chamber after washing. Because of the limited field of view of the objective used, the image is stitched together from two individual images after acquisition. The microarray was visualized by an epifluorescense microscope equipped with proper filters and a camera. The experiment demonstrates that high quality microarray analysis can be conducted using a closed microarray platform as shown in FIG. 1.

TABLE 2

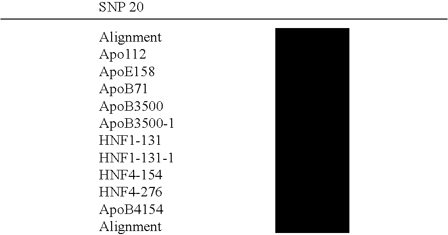

What is claimed is:

1. A closed substrate platform comprising:
a slide element enclosed within a container; wherein,
the slide comprises a defined microfluidic analysis platform; wherein,
the container comprises at least one inlet port for introduction of liquid into the container which delivers liquid to the area for sample analysis; wherein, the container comprises at least one vent for expulsion of air from the container; and,
the container comprises an outlet port for removal of liquid from the area for sample analysis.

2. The closed substrate platform of claim 1, wherein the sample or buffer is introduced into the platform via an inlet port.

3. The closed substrate platform of claim 2, wherein the inlet port is conical.

4. The closed substrate platform of claim 2, wherein the inlet port is connected to a horizontal small chamber directly beneath the inlet port.

5. The closed substrate platform of claim 4, wherein the horizontal chamber is connected to a vertical chamber.

6. The closed substrate platform of claim 5, wherein the vertical chamber is connected to the horizontal chamber at an angle of at least about 90°.

7. The closed substrate platform of claim 6, wherein the vertical chamber is connected to the bottom of an inlet chamber.

8. The closed substrate platform of claim 5, wherein the vertical chamber is positioned parallel to the inlet port, such that, the sample introduced via the inlet port flows through a U-shape.

9. The closed substrate platform of claim 7, wherein the length of the inlet chamber is at least about 10 mm to at least about 25 mm.

10. The closed substrate platform of claim 9, wherein the width of the inlet chamber is at least about 1 mm to at least about 3 mm

11. The closed substrate platform of 7, wherein the inlet chamber is connected to a U-shaped channel.

12. The closed substrate platform of claim 11, wherein the U-shaped channel is comprised of at least two parallel channels connected via at least about two 180° semi-circular turns.

13. The closed substrate platform of claim 12, wherein the U-shaped channel is connected to the substrate analysis platform.

14. The closed substrate platform of claim 13, wherein the substrate analysis platform is of a meandering design.

15. The closed substrate platform of claims 1 or 14, wherein the substrate analysis platform is comprised of a meandering design comprising at least about one U-shaped channel.

16. The closed substrate platform of claim 15, wherein the meandering substrate platform is comprised of at least two parallel running stretches of tubes connected via a 180° semi-circular end to at least about ten parallel running stretches of tubes connected via a 180° semi-circular end.

17. The closed substrate platform of claim 1, wherein the volume of sample required for analysis is at least about 40:1 to at least about 200:1.

18. The closed substrate platform of claim 1, wherein the substrate analysis platform is comprised of two sections; wherein,
one section, a top section, is comprised of desired immobilized biomolecules; and,
another section, a bottom section, comprising the microfluidics structure of the analysis platform.

19. The closed substrate platform of claim 18, wherein the biomolecules immobilized onto the one section are positioned directly above the microfluidics structure of the analysis platform.

20. The closed substrate platform of claim 18, wherein the top section is placed over the microfluidics structure of the bottom section and sealed.

21. The closed substrate platform of claim 20, wherein the top and bottom section of the substrate analysis platform are sealed using transmission laser welding, and the like.

22. The closed substrate platform of claim 20, wherein the seal is airtight.

23. The closed substrate platform of claim 1, wherein the outlet for removal of liquid is connected to a waste chamber.

24. The closed substrate platform of claim 1, wherein the waste area holds at least about 1 ml of liquid.

25. The closed substrate platform of claim 23, wherein the waste area further contains an absorbent material.

26. The closed substrate platform of claim 25, wherein the absorbent material is a gel.

27. A closed substrate platform comprising:
a slide element enclosed within a container, preferably a sealed container with an opening that can provide fluid communication to the system, and the slide element preferably being plastic,
wherein the slide comprises a defined area for sample analysis,
wherein the container comprises at least one inlet port for introduction of liquid into the sealed container which delivers liquid to the area for sample analysis preferably via a tube or channel,
wherein the sample analysis area is of a meandering design,
wherein the sealed container comprises at least one vent for expulsion of air from the sealed container,
wherein the sealed container comprises an outlet port for removal of liquid from the area for sample analysis preferably via a tube, channel, or the like.

28. A closed substrate platform comprising:
a slide element enclosed within a container, preferably a sealed container with an opening that can provide fluid communication to the system, and the slide element preferably being plastic,
wherein the slide comprises a defined area for sample analysis,
wherein the sample analysis area is of a meandering design,
wherein the container comprises a first inlet port for introduction of a sample into the sealed container and which delivers the sample to the area for sample analysis preferably via a tube or channel,
wherein the container comprises a first vent for expulsion of air from the sealed container due to introduction of sample through the first inlet port,
wherein the sealed container comprises a second inlet port for introduction of a fluid such as a wash buffer into the container and which delivers the fluid (preferably wash buffer) to the area for sample analysis preferably via a tube or channel, and
wherein the container comprises a second vent for expulsion of air from the container due to introduction of fluid through the second inlet port.

29. A closed substrate platform comprising:
a slide element enclosed within a container, preferably a sealed container with an opening that can provide fluid communication to the system, and the slide element preferably being plastic,
wherein the slide comprises a defmed area for sample analysis,
wherein the container comprises at least one inlet port for introduction of liquid into the sealed container which delivers liquid to the area for sample analysis preferably via a tube or channel,
wherein the sample analysis area is a straight channel,
wherein the inlet port is fitted with a rubber or silicon-based adaptor for introduction of a sample into the sealed container; and,
said adaptor is conically shaped to allow for introduction of the sample into the adaptor with a minimized risk of spillage or back flow,
wherein the fluid travels from the adapter to the analysis area through a canal which is interrupted by a flow restrictor, afterwhich,
the fluid travels from the analysis area through a canal and arrives at a buffer chamber which is connected to a short capillary canal that opens into a waste area,
wherein, the waste area ends in a vent, shaped as a capillary chamber.

30. The closed substrate platform of claim 29, wherein the straight narrow channel preferably is at least about 1 mm wide and about 3 mm wide.

31. The closed substrate platform of claim 29, wherein comprises at least one individual straight narrow channel to about five individual straight narrow channels.

32. The closed substrate platform of claim 27, 28, or 29 further comprising a waste area to receive fluid from the sample analysis area.

33. The closed substrate platform of claim 32, wherein the waste area holds at least about 2 ml of fluid.

34. The closed substrate platform of claim 32, wherein the waste area holds at least about less than 1 ml of fluid.

35. The closed substrate platform of claim 33 or 34 wherein the waste area is comprised of a meandering design.

36. The closed substrate platform of claim 35, wherein the waste area is connected to a vent in the shape of a capillary tube.

37. The closed substrate platform of any one of claims 1, 27 through to 29, wherein the inlet ports and vents comprise a resealable septum.

38. The substrate platform of any one of claims 1, 27 through to 29, wherein the substrate platform further comprises a mark used to identify the slide.

39. The substrate platform of claim 38, wherein the mark is a bar code.

40. The substrate platform of claims 1, 27 through to 29, wherein the slide is constructed from one or more polymer materials of polycarbonate or Topas (COC) or COP.

41. The substrate platform of claims 1, 27 through to 29, wherein the surface of the slide is treated so as to increase the binding capacity of the slide.

42. The substrate platform of claims 1, 27 through to 29, wherein the slide is constructed of a material which is resistant to temperatures over a range of −5° C. to +105° C.

43. The substrate platform of claims 1, 27 through to 29, wherein the slide is constructed of a material which is resistant to pH over a range of pH=1 to pH=13.

44. The substrate platform of claims 1, 27 through to 29, which is dimensioned so as to be compatible with equipment capable of handling a standard microscope slide.

45. The substrate platform of claim 29, wherein the dimensions of the slide element are at least about 75 mm to at least about 76 mm long, at least about 24 mm to at least about 25 mm wide, and at least about 1 mm to at least about 2 mm thick.

46. The substrate platform of claim 45, wherein the slide element is at least about 1 mm thick.

47. The substrate platform of claims 1, 27 through to 29, which is constructed using injection molding.

48. The substrate platform of claims 1, 27 through to 29, which further comprises immobilized nucleic acid sequences.

49. The substrate platform of claim 48, wherein the nucleic acid sequences are modified.

50. The substrate platform of claim 49, wherein the nucleic acid sequences contain at least one modified nucleotide.

51. The substrate platform of claim 49, wherein the nucleic acid sequences contain at least one locked nucleoside analogue.

52. The substrate platform of claim 49, wherein the nucleic acid sequences are completely composed of locked nucleoside analogues.

53. The substrate platform of claim 49, wherein the nucleic acid sequences contain at least one modified internucleoside linkage.

54. The substrate platform of claim 49, wherein the nucleic acid sequences contain at least one phosphorothioate internucleoside linkage.

55. The substrate platform of claim 49, wherein all of the internucleoside linkages of the nucleic acid sequences are phosphorothioate.

56. The substrate platform of claim 49, wherein the nucleic acid sequences comprise at least one modified nucleotide and at least on modified internucleoside linkage.

57. The substrate platform of claim 48, wherein each immobilized nucleic acid with a unique sequence is located at a defined position.

58. The substrate platform of claim 57, which comprises at least 100 unique sequences per cm².

59. The substrate platform of claim 57, which comprises at least 400 unique sequences per cm².

60. The substrate platform of claim 57, which comprises at least 900 unique sequences per cm².

61. The substrate platform of claim 57, wherein each immobilized nucleic acid contains from about 500 to about 1000 nucleotides.

62. The substrate platform of claim 57, wherein each immobilized nucleic acid contains from about 100 to about 500 nucleotides.

63. The substrate platform of claim 57, wherein each immobilized nucleic acid contains from about 10 to about 100 nucleotides.

64. The substrate platform of claim 57, wherein each immobilized nucleic acid contains from about 2 to about 30 nucleotides.

65. The substrate platform of claim 48, wherein the nucleic acid sequences are immobilized onto the slide using a photochemical linker.

66. The substrate platform of claim 65, wherein the nucleic acid sequences are immobilized onto the slide using anthraquinone.

67. The substrate platform of claim 48, wherein a linker connects either the 5′ or 3′ ends of the nucleic acid sequences to the surface of the slide.

68. The substrate platform of claim 48, wherein the nucleic acid sequences are immobilized onto the surface of the slide after synthesis.

69. The substrate platform of claim 48, wherein the nucleic acid sequences are synthesized on the surface of the slide.

70. The substrate platform of claim 48, wherein the nucleic acid sequences are double stranded.

71. The substrate platform of claim 48, wherein the nucleic acid sequences are single stranded.

72. The substrate platform of any one of claims 1 through 71 which further comprises immobilized polypeptides.

73. The substrate platform of claim 72, wherein the immobilized polypeptides contains at least one modification selected from the group consisting of phosphorylation or glycosylation.

74. The substrate platform of claim 72, wherein each immobilized polypeptide with a different amino acid sequence is located at a defined position.

75. The substrate platform of claim 74, which comprises at least 100 unique polypeptide sequences per cm².

76. The substrate platform of claim 74, which comprises at least 400 unique polypeptide sequences per cm².

77. The substrate platform of claim 74, which comprises at least 900 unique polypeptide sequences per cm².

78. The substrate platform of claim 72, wherein the polypeptides are immobilized onto the slide using a photochemical linker.

79. The substrate platform of claim 72, wherein the polypeptides are immobilized onto the slide using anthraquinone.

80. The substrate platform of claim 72, wherein a flexible linker connects either the amino-termini or carboxy-termini of the polypeptides to the surface of the slide.

81. The substrate platform of claim 72, wherein the polypeptides are synthesized on the surface of the slide.

82. The substrate platform of claims 27 through to 29, wherein the analysis area is modified to facilitate attachment and growth of cells.

83. A method for identifying a nucleic acid sequence capable of binding to a biomolecule comprising:
immobilizing each unique nucleic acid sequence from a library of nucleic acid sequences onto the substrate platform of claims 1, 27 through to 29,
optionally washing the substrate platform to remove contaminants,
incubating the immobilized nucleic acid sequences with a biomolecule under conditions which are conducive to specific interaction between the biomolecule and the nucleic acid sequences,
optionally washing the substrate platform to remove any non-specifically bound biomolecules,
detecting the location of the nucleic acid sequences which bound to the biomolecule.

84. The method of claim 63, wherein the biomolecule is a nucleic acid sequence.

85. The method of claim 63, wherein the biomolecule is a polypeptide.

86. The method of claim 63, wherein the location of the nucleic acid sequences which bound to the biomolecule is detected by virtue of a tag on the biomolecule.

87. The method of claim 86, wherein the tag on the biomolecule is a detectable moiety.

88. A method for identifying a polypeptide capable of binding to a biomolecule comprising:
immobilizing each unique polypeptide from a library of polypeptides onto the substrate platform of claims 1, 27 through to 29,
optionally washing the substrate platform to remove contaminants,
incubating the immobilized polypeptides with a biomolecule under conditions which are conducive to specific interaction between the biomolecule and the polypeptides,
optionally washing the substrate platform to remove any non-specifically bound biomolecules,
detecting the location of the polypeptides which bound to the biomolecule.

89. The method of claim 88, wherein the biomolecule is a nucleic acid sequence.

90. The method of claim 89, wherein the biomolecule is a polypeptide.

91. The method of claim 89, wherein the biomolecule is a multimeric polypeptide.

92. The method of claim 89, wherein the biomolecule is an antibody.

93. The method of claim 89, wherein the biomolecule is a receptor.

94. The method of claim 89, wherein the biomolecule is a hormone.

95. The method of claim 89, wherein the biomolecule is a drug or drug candidate.

96. The method of claim 89, wherein the location of the polypeptides which bound to the biomolecule is detected by virtue of a tag on the biomolecule.

97. The method of claim 89, wherein the tag on the biomolecule is a fluorescent tag.

98. A method for sample analysis comprising:
applying a sample to the substrate platform of claims 1, 27 through to 29; and,
evaluating the sample.

99. The method of claim 98, wherein the sample is a liquid.

100. Use of the substrate platform of any one of claims 1, 27 through to 29, for sample analysis.

101. Use of the substrate platform of any one of claims 1, 27 through to 29, for detecting DNA sequence variation, DNA sequencing, SNP analysis, genotyping, deletion analysis, gene expression and the like.

102. A method for producing the substrate platform of any one of claims 1, 27 through to 29; wherein,
the slide is comprised of a bottom surface plastic structure, and
the top surface of the slide is comprised of a thin plastic film or laminate; wherein,
said film or laminate is placed over the bottom part of the slide and sealed using heat or adhesive followed by physical pressure to ensure airtight sealing and prevent any liquid or gas from escaping through the seal.
